(19) United States
(12) Patent Application Publication
(10) Pub. No.: US 2015/0225309 A1
(43) Pub. Date: Aug. 13, 2015

(54) METHOD OF PRODUCING OLEFIN HAVING 2 TO 4 CARBON ATOMS AND METHOD OF PRODUCING PROPYLENE

(71) Applicants: SUMITOMO CHEMICAL COMPANY, LIMITED, Tokyo (JP); National University Corporation University of Toyama, Toyama-shi, Toyama (JP)

(72) Inventors: Noritatsu Tsubaki, Toyama-shi (JP); Takeshi Ishiyama, Tsukuba-shi (JP); Yusuke Shibata, Tsukuba-shi (JP); Hideyuki Higashimura, Tsukuba-shi (JP); Akihiro Yuasa, Tokyo (JP)

(73) Assignees: SUMITOMO CHEMICAL COMPANY, LIMITED, Tokyo (JP); National University Corporation University of Toyama, Toyama-shi, Toyama (JP)

(21) Appl. No.: 14/419,722

(22) PCT Filed: Aug. 1, 2013
(86) PCT No.: PCT/JP2013/070882

§ 371 Date: Feb. 5, 2015

(30) Foreign Application Priority Data
Aug. 10, 2012 (JP) 2012-178547
Feb. 28, 2013 (JP) 2013-040103

(51) Int. Cl. C07C 1/06; C07C 4/06; C07C 1/04

(52) U.S. Cl. CPC C07C 1/06; C07C 1/044; C07C 4/06; C07C 1/043; C07C 2521/18; C07C 2529/46; C07C 2529/40; C07C 2529/85; C07C 2523/02; C07C 2523/889; C07C 2523/72

(57) ABSTRACT

A method of producing olefin having 2 to 4 carbon atoms, including a process of reacting at least one kind of a catalyst (D) selected from the group consisting of below catalysts (A) to (C) with synthesis gas in the presence of a dispersion medium through a Fischer-Tropsch reaction, in which the catalyst (A) contains iron and one to three kinds of elements selected from the group consisting of alkali metal and alkali earth metal, the catalyst (B) contains cobalt, provided that the catalyst (B) is a catalyst except a catalyst obtained by reducing a cobalt ion and an iron ion in a dispersion liquid or a solution containing the cobalt ion, the iron ion and a dispersant that interacts with the cobalt ion and the iron ion, and the catalyst (C) contains nickel or ruthenium.

### TECHNICAL FIELD

The present invention relates to a method of producing olefin having 2 to 4 carbon atoms through a Fischer-Tropsch reaction (hereinafter, referred to as “light olefin” in some cases), and a method of producing propylene.

Priorities are claimed on Japanese Patent Application No. 2012-178547, filed on Aug. 10, 2012, and Japanese Patent Application No. 2013-040103, filed on Feb. 28, 2013, the contents of which are incorporated herein by reference.

### BACKGROUND ART

A Fischer-Tropsch reaction (hereinafter, also referred to as an “FT reaction”) is known as a reaction for synthesis of a hydrocarbon from a mixture of carbon monoxide and hydrogen (hereinafter, also referred to as “synthesis gas”). The FT reaction is a reaction using a metal catalyst, and represented by the following Chemical Formula (1).

nCO+2nH₂→(CH₂)ₙ+nH₂O (1)

In the related art, the objective product of synthesis of a hydrocarbon by conducting the FT reaction is a saturated hydrocarbon in most cases. Such a saturated hydrocarbon has been used as fuel or a lubricant after being subjected to various processes such as hydrogenolysis or isomerization.

In the FT reaction in the related art, an unsaturated hydrocarbon or an oxygen-containing compound such as alcohol may be generated at the same time when the saturated hydrocarbon is generated. However, the selectivity of these compounds in the FT reaction in the related art is exceedingly low.

On the other hand, lower olefin (i.e., olefin having lower carbon atoms) such as ethylene, propylene and butene has been widely used as a raw material compound. For example, propylene is used as a stating material for producing polypropylene. In recent years, production of olefin using the FT reaction has been considered.

For example, Patent Literatures 1 and 2 disclose an FT reaction to be conducted for a purpose of producing olefin with high yield, in which an iron-based catalyst having a manganese compound is used as a support.

### PRIOR ART DOCUMENT

### Patent Literature

[Patent Literature 1] Japanese Examined Patent Application Publication No. S56-48491

[Patent Literature 2] U.S. Pat. No. 4,177,203

### SUMMARY OF THE INVENTION

### Problems to be Solved by the Invention

However, the catalyst and the method disclosed in Patent Literatures 1 and 2 cannot be said to be necessarily sufficient from the viewpoint of the content ratio of an unsaturated hydrocarbon, particularly light olefin having 2 to 4 carbon atoms (hereinafter, also referred to as “C2 to C4 olefin”) contained in reaction products, so that the improvement thereof is desired.

Further, in the description below, the content ratio of an unsaturated hydrocarbon contained in the reaction products is also referred to as the “selectivity of an unsaturated hydrocarbon.” Furthermore, the content ratio of C2 to C4 olefin contained in the reaction products is also referred to as the “selectivity of C2 to C4 olefin.” Similar to this, the content ratio of a specific compound (for example, propylene) contained in the reaction products is also referred to as the “selectivity of the compound.”

The present invention has been developed in light of the circumstances described above, and has an object of providing a method of producing light olefin having 2 to 4 carbon atoms capable of obtaining high selectivity, and particularly to provide a method of producing lower olefin with high selectivity of propylene.

### Means for Solving the Problem

In order to solve the problems, an aspect of the present invention will be described as follows.

[1] A method of producing olefin having 2 to 4 carbon atoms, including a process of reacting at least one kind of a catalyst (D) selected from the group consisting of catalysts (A) to (C) with synthesis gas in the presence of a dispersion medium through a Fischer-Tropsch reaction, in which the catalyst (A) contains iron and one to three kinds of elements selected from the group consisting of alkali metal and alkali earth metal, the catalyst (B) contains cobalt, provided that the catalyst (B) is a catalyst except a catalyst obtained by reducing a cobalt ion and an iron ion in a dispersion liquid or a solution containing the cobalt ion, the iron ion a dispersant that interacts with the cobalt ion and the iron ion, and the catalyst (C) contains nickel or ruthenium.

[2] The method of producing olefin having 2 to 4 carbon atoms according to item [1], in which the catalyst (D) contains one to three kinds of elements selected from the group consisting of manganese, copper, zinc, titanium, zirconium, lanthanum, and cerium.

[3] The method of producing olefin having 2 to 4 carbon atoms according to item [1] or [2], in which the catalyst (D) contains elements (1) and elements (2), and satisfies a condition (3), the elements (1) are iron and manganese, the elements (2) are one to three kinds of metal elements selected from the group consisting of alkali metal and alkali earth metal, and the condition (3) is 50≦a≦90, 9.5≦b≦48, 0.5≦c≦10, provided that a+b+c=100, when the molar ratio of iron is represented by a mole %, the molar ratio of manganese is represented by b mole %, and the molar ratio of the total metal elements in the elements (2) is represented by c mole %, relative to the total number of moles of the iron, the manganese and the elements (2).

[4] The method of producing olefin having 2 to 4 carbon atoms according to any one of items [1] to [3], in which the catalyst (D) further contains a carbon support.

[5] The method of producing olefin having 2 to 4 carbon atoms according to any one of items [1] to [4], in which the synthesis gas contains hydrogen and carbon monoxide, and the molar ratio of the hydrogen relative to the carbon monoxide, which is represented by “hydrogen/carbon monoxide”, is in the range of from 0.3 to 3.

[6] The method of producing olefin having 2 to 4 carbon atoms according to any one of items [1] to [5], in which the reaction temperature in the process of reacting the synthesis gas with the catalyst (D) is in the range of from 100° C. to 600° C.

[7] The method of producing olefin having 2 to 4 carbon atoms according to any one of items [1] to [6], in which the reaction pressure in the process of reacting the synthesis gas with the catalyst (D) is in the range of from 0.1 MPa to 50 MPa.

[8] The method of producing olefin having 2 to 4 carbon atoms according to any one of items [1] to [7], in which the dispersion medium is an organic compound which becomes a liquid state in the temperature range of from 100° C. to 600° C. under the normal pressure.

[9] The method of producing olefin having 2 to 4 carbon atoms according to any one of items [1] to [8], in which the ratio of the total number of carbon atoms constituting olefin having 2 to 4 carbon atoms relative to the total number of carbon atoms constituting a hydrocarbon product obtained from the process of reacting the synthesis gas with the catalyst (D) is 18% or more.

[10] The method of producing olefin having 2 to 4 carbon atoms according to any one of items [1] to [9], further including a process of catalytically cracking the product obtained from the process of reacting the synthesis gas with the catalyst (D), after the process of reacting the synthesis gas with the catalyst (D).

[11] A method of producing propylene which uses the method of producing olefin having 2 to 4 carbon atoms according to any one of items [1] to [10].

Another aspect of the present invention will be described as follows.

[12] A method of producing olefin having 2 to 4 carbon atoms, including: a first process of reacting synthesis gas and a catalyst (E) in the presence of a dispersion medium to produce a hydrocarbon product through a Fischer-Tropsch reaction; and a second process of catalytically cracking the hydrocarbon product by allowing the hydrocarbon product to come into contact with a cracking catalyst which is consisting of zeolite containing one or more kinds of elements selected from the group consisting of alkali metal, alkali earth metal and transition metal.

[13] The method of producing olefin having 2 to 4 carbon atoms according to item [12], in which the zeolite contains one or more kinds of elements selected from the group consisting of alkali metal, alkali earth metal and a d-block element.

[14] The method of producing olefin having 2 to 4 carbon atoms according to item [12] or [13], in which the zeolite is ZSM-5, and the molar ratio of SiO₂ relative to Al₂O₃ in the zeolite, which is represented by “SiO₂/Al₂O₃”, is in the range of from 50 to 4000.

[15] The method of producing olefin having 2 to 4 carbon atoms according to any one of items [12] to [14], in which the cracking catalyst contains one or more kinds of elements selected from the group consisting of the alkali metal, the alkali earth metal and the transition metal, of which the content of the elements is in the range of from 0.01% by mass to 30% by mass relative to the total mass of the cracking catalyst.

[16] The method of producing olefin having 2 to 4 carbon atoms according to any one of items [12] to [15], in which one or more kinds of elements selected from the group consisting of the alkali metal, the alkali earth metal and the transition metal contained in the cracking catalyst is alkali earth metal.

[17] The method of producing olefin having 2 to 4 carbon atoms according to any one of items [12] to [16], in which the reaction pressure in the catalytic cracking is in the range of from 0.01 MPa to 0.5 MPa.

[18] The method of producing olefin having 2 to 4 carbon atoms according to any one of items [12] to [17], in which the catalyst (E) contains at least one kind of element selected from the group consisting of iron, cobalt, nickel, and ruthenium.

[19] The method of producing olefin having 2 to 4 carbon atoms according to item [18], in which the catalyst (E) further contains one to three kinds of elements selected from the group consisting of manganese, copper, zinc, titanium, zirconium, lanthanum and cerium.

[20] The method of producing olefin having 2 to 4 carbon atoms according to item [18] or [19], in which the catalyst (E) further contains one to three kinds of elements selected from the group consisting of alkali metal and alkali earth metal.

[21] The method of producing olefin having 2 to 4 carbon atoms according to any one of items [12] to [20], in which the catalyst (E) contains elements (1) and elements (2), and satisfies a condition (3), in which the elements (1) are iron and manganese, the elements (2) are one to three kinds of metal elements selected from the group consisting of alkali metal and alkali earth metal, and the condition (3) is 50≦a≦90, 9.5≦b≦48, and 0.5≦c≦10, provided that a+b+c=100, when the molar ratio of iron is represented by a mole %, the molar ratio of manganese is represented by b mole %, and the molar ratio of the total metal elements in the elements (2) is represented by c mole %, relative to the total number of moles of the iron, the manganese and the elements (2).

[22] The method of producing olefin having 2 to 4 carbon atoms according to any one of items [12] to [21], in which the catalyst (E) further contains a carbon support.

[23] The method of producing olefin having 2 to 4 carbon atoms according to any one of items [12] to [22], in which the synthesis gas contains hydrogen and carbon monoxide, and the molar ratio of the hydrogen relative to the carbon monoxide, which is represented by “hydrogen/carbon monoxide”, is in the range of from 0.3 to 3.

[24] The method of producing olefin having 2 to 4 carbon atoms according to any one of items [12] to [23], in which the reaction temperature in the first process is in the range of from 100° C. to 600° C.

[25] The method of producing olefin having 2 to 4 carbon atoms according to any one of items [12] to [24], in which the reaction pressure in the first process is in the range of from 0.1 MPa to 50 MPa.

[26] The method of producing olefin having 2 to 4 carbon atoms according to any one of items [12] to [25], in which the dispersion medium is an organic compound which becomes a liquid state in the temperature range of from 100° C. to 600° C. under the normal pressure.

[27] The method of producing olefin having 2 to 4 carbon atoms according to any one of items [12] to [26], in which the ratio of the total number of carbon atoms constituting olefin having 2 to 4 carbon atoms relative to the total number of carbon atoms constituting the hydrocarbon product obtained from the first process is 18% or more.

[28] A method of producing propylene by using the method of producing olefin having 2 to 4 carbon atoms according to any one of items [12] to [27].

Further, the common feature of the first and second aspects of present invention is as follows.

[29] A method of producing olefin having 2 to 4 carbon atoms, including a process of reacting synthesis gas with a catalyst which contains at least one kind of element selected from the group consisting of iron, cobalt and nickel and contains one to three kinds of elements selected from the group consisting of alkali metal and alkali earth metal, in the presence of a dispersion medium through a Fischer-Tropsch reaction.

### Effect of the Invention

According to the aspects of the present invention, a method of producing olefin having 2 to 4 carbon atoms capable of obtaining high selectivity, and particularly a method of producing olefin having high selectivity of propylene can be provided.

### BRIEF DESCRIPTION OF THE DRAWING

FIG. 1 is a schematic diagram illustrating an example of a production apparatus embodying a method of producing olefin having 2 to 4 carbon atoms according to a second embodiment.

### MODES FOR CARRYING OUT THE INVENTION

### First Embodiment

A method of producing olefin having 2 to 4 carbon atoms according to the present embodiment includes a process of reacting synthesis gas with at least one kind of catalyst (D) selected from the group consisting of the below catalysts (A) to (C) in the presence of a dispersion medium through a Fischer-Tropsch reaction (hereinafter, also referred to as a “FT reaction”).

Catalyst (A): a catalyst containing iron and one to three kinds of elements selected from the group consisting of alkali metal and alkali earth metal.

Catalyst (B): a catalyst containing cobalt (provided that the catalyst (B) excludes a catalyst obtained by reducing a cobalt ion and an iron ion in a dispersion liquid or a solution containing the cobalt ion and the iron ion and a dispersant that interacts with the cobalt ion and the iron ion).

Catalyst (C): a catalyst containing nickel or ruthenium.

In addition, in the present specification, examples of the olefin having 2 to 4 carbon atoms include ethylene, propylene, 1-butene, 2-butene, isobutene, and 1,3-butadiene.

(Catalyst (D))

The catalyst (A) contains one to three kinds of elements selected from the group consisting of alkali metal and alkali earth metal, and the elements function as a promotor. Examples of the elements preferably include lithium, sodium, potassium, rubidium, cesium, beryllium, magnesium, calcium, strontium, and barium; more preferably sodium, potassium, rubidium, cesium, magnesium, calcium, strontium, and barium; still more preferably sodium, potassium, magnesium, and calcium; and particularly preferably potassium and magnesium.

The catalyst (B) excludes a catalyst obtained by reducing a cobalt ion and an iron ion in a dispersion liquid or a solution containing the cobalt ion and the iron ion, and the dispersant that interacts with the cobalt ion and the iron ion.

That is, the catalyst (B) does not include a catalyst obtained from a production method including: preparing the dispersion liquid or the solution containing the cobalt ion, the iron ion and the dispersant that interacts with the cobalt ion and the iron ion; and reducing the cobalt ion and the iron ion by adding a reducing agent to the dispersion liquid or the solution.

With respect to the catalyst to be excluded, “the dispersant that interacts with the cobalt ion and iron ion” prevents the aggregation of generated alloy particles in the above-described dispersion liquid or the solution during the reduction reaction or after the reduction reaction (that is, the reacted liquid).

Examples of a water-soluble polymer among the dispersant include a polymer having an alkylene ether structure such as polyethylene glycol (PEG), and polypropylene glycol; polyvinyl alcohol; polyvinyl ether; polyacrylate; polyvinyl pyrrolidone (PVP); poly(mercaptomethylenestyrene-N-vinyl-2-pyrrolidone); and polyacrylonitrile.

Examples of the solvent used for the preparation of “the dispersion liquid or the solution” include alcohol such as 1,2-ethanediol (ethylene glycol), 1,2-propanediol, 1,3-propanediol, 1,2-butanediol, 1,3-butanediol, 1,4-butanediol, 2,3-butanediol, pentanediol, hexanediol, heptanediol, octanediol, diethylene glycol, triethylene glycol, tetraethylene glycol, dipropylene glycol, hexylene glycol, 2-butene-1,4-diol, glycerol, 1,1,1-trishydroxymethylethane, 2-ethyl-2-hydroxymethyl-1,3-propanediol, 1,2,3-hexanetriol and benzyl alcohol.

Further, “the dispersion liquid or the solution” can be prepared by blending metal-containing compounds which are ion sources of an cobalt ion and an iron ion, a dispersant, a solvent, and other components (for example, a reducing agent described later) as needed.

The concentration of the dispersant in the dispersion liquid or the solution is for example, 1×10⁻⁴% by mass to 5% by mass relative to the total mass of the dispersion liquid or the solution.

A known reducing agent is used for the reduction of the cobalt ion and the iron ion, and examples thereof include sodium borohydride (NaBH₄), potassium borohydride (KBH₄), sodium triethylborohydride (Na(CH₃CH₂)₃BH), potassium triethylborohydride (K(CH₃CH₂)₃BH), sodium cyanoborohydride (NaBH₃CN), lithium borohydride (LiBE₄), lithium triethylborohydride (LiBH(CH₂CH₃)₃) and triethylsilane (CH₃CH₂)₃SiH.

The blending amount of the reducing agent is, for example, 0.1 moles or more per one mole of a metal ion to be reduced.

The reaction temperature during the reduction of the cobalt ion and the iron ion is, for example, 20° C. to 200° C., and the reaction time thereof is, for example, 1 to 120 minutes.

It is assumed that the catalyst obtained in this way does not correspond to the catalyst (B).

Further, the catalyst (A) may further contain one or more metal elements selected from the group consisting of cobalt, nickel and ruthenium.

Furthermore, the catalyst (B) may further contain one or more metal elements selected from the group consisting of iron, alkali metal, alkali earth metal, nickel and ruthenium.

Furthermore, the catalyst (C) may further contain one or more metal elements selected from the group consisting of iron, alkali metal, alkali earth metal and cobalt. Furthermore, the catalysts (A) to (C) may be used in combination.

The catalysts (A) to (C) may contain one to three kinds of other transition metal elements as a promotor. The transition metal element is preferably manganese, copper, zinc, titanium, zirconium, lanthanum or cerium, more preferably manganese or copper, and particularly preferably manganese.

When the catalyst (A) contains the transition metal element as a promotor, the content of iron is preferably 50 mole % to 90 mole %, the total content of alkali metal and alkali earth metal is preferably 0.5 mole % to 10 mole %, and the total content of the transition metal element as a promotor is preferably 9.5 mole % to 48 mole %, relative to the total number of moles of the iron, the alkali metal, the alkali earth metal and the transition metal element as a promotor. More preferably, the content of iron is 50 mole % to 90 mole %, the total content of alkali metal and alkali earth metal is 0.5 mole % to 10 mole %, and the total content of the transition metal element as a promotor is 9.5 mole % to 45 mole %.

When the catalyst (B) contains the transition metal element as a promotor, the mass ratio of the transition metal element as a promotor relative to cobalt, which is represented by “the total of the transition metal element as a catalyst/cobalt”, is preferably in the range of from 0.01 to 5.

When the catalyst (C) contains the transition metal element as a promotor, the mass ratio of the transition metal element as a promotor relative to nickel or ruthenium, which is represented by “the total of the transition metal element as a promotor/nickel or ruthenium”, is preferably in the range of from 0.01 to 5.

The catalyst (D) used for the method of producing C2 to C4 olefin of the present embodiment is preferably the catalyst (A) or the catalyst (B), and more preferably the catalyst (B) or the catalyst (A) further containing manganese, that is, the catalyst containing the elements (1) which are iron and manganese and the elements (2) which are one to three kinds of elements selected from the group consisting of an alkali metal element and an alkali earth metal element.

The catalyst (A) contains iron, so that the reactivity of the FT reaction can be easily secured, which is preferable.

In addition, a catalyst may contain cobalt or copper other than those described above. When copper is included in the catalyst, the reduction of iron is accelerated in the activation treatment described later, which is preferable.

The elements (2) preferably include lithium, sodium, potassium, rubidium, cesium, beryllium, magnesium, calcium, strontium, and barium; more preferably sodium, potassium, rubidium, cesium, magnesium, calcium, strontium and barium; still more preferably sodium, potassium, magnesium and calcium; and particularly preferably potassium and magnesium.

In addition, when the elements (2) contained in the catalyst (A) are magnesium, a gas shift reaction (a reaction of generating carbon dioxide and hydrogen by reacting carbon monoxide and water) which is a competition reaction of the FT reaction can be suppressed, which is preferable.

With respect to the molar ratio of the elements (1) relative to the metal elements in the element (2) included in the catalyst (A), when the molar ratio of iron is represented by a mole %, the molar ratio of manganese is represented by b mole %, and the total molar ratio of the metal element in the elements (2) is represented by c mole % relative to the total number of moles of the iron, the manganese and the total number of metal elements of the element (2), the molar ratio is preferably 50≦a≦90, 9.5≦b≦48, and 0.5≦c≦10, (provided that a+b+c=100), and more preferably 50≦a≦90, 9.5≦b≦45, and 0.5≦c≦10(provided that a+b+c=100).

The selectivity of C2 to C4 olefin is increased by controlling the molar ratio of the catalyst in the range.

The molar ratio of the elements (1) relative to the elements (2) included in the catalyst (A) is still more preferably 55≦a≦85, 9.5≦b≦45, and 1≦c≦7, (provided that a+b+c=100), and further still more preferably 60≦a≦80, 15≦b≦40, and 1≦c≦6, (provided that a+b+c=100).

The catalyst (B) contains cobalt, so that the gas shift reaction can be suppressed, which is preferable.

The catalyst (B) may include manganese, zinc, or the like in addition to cobalt. When manganese or zinc is included in the catalyst (B), the olefin ratio in a hydrocarbon generated by the FT reaction is increased, which is preferable.

The amount of manganese contained in the catalyst (B) is preferably in the range of from 0.01 times to 5 times (by mass), more preferably in the range of from 0.1 times to 4 times (by mass), and still more preferably in the range of from 0.5 times to 4 times (by mass) relative to the amount of cobalt. Further, the amount of zinc is preferably in the range of from 0.01 times to 5 times (by mass), more preferably in the range of from 0.01 times to 1 time (by mass), and still more preferably in the range of from 0.01 times to 0.2 times (by mass) relative to the content of cobalt.

The catalyst (D) used for the method of producing C2 to C4 olefin of the present embodiment may be a catalyst containing the following elements (3) and (4).

Elements (3): at least one kind of element selected from the group consisting of iron, cobalt and nickel.

Elements (4): one to three kinds of elements selected from the group consisting of alkali metal and alkali earth metal.

The molar ratio of the elements (3) to the elements (4) included in the catalyst (D), which is represented by “the total of the elements (3)/the total of the elements (4)”, is preferably 5 to 180. The reactivity of the FT reaction is easily secured by controlling the molar ratio of the catalyst in this way.

The catalyst (D) used for the method of producing C2 to C4 olefin of the present embodiment is preferably a combination of iron and potassium as catalytic metal, and the molar ratio thereof, which is represented by “iron/potassium”, is preferably 5 to 180. Moreover, the catalyst (D) may further contain manganese, in this case, the content of iron is preferably 50 mole % to 90 mole %, the content of manganese is preferably 9.5 mole % to 48 mole %, and the content of potassium is preferably 0.5 mole % to 10 mole % relative to the total number of moles of the iron, the manganese and the potassium; the content of iron is more preferably 50 mole % to 90 mole %, the content of manganese is more preferably 9.5 mole % to 45 mole %, and the content of potassium is more preferably 0.5 mole % to 10 mole % relative to the total number of moles of the iron, the manganese, and the potassium.

Further, the molar ratio of metal contained in a catalyst in the present embodiment may be determined by using Energy Dispersive X-ray Fluorescence Spectrometry (hereinafter, also referred to as “EDS Spectrometry”) or Inductively Coupled Plasma Emission Spectrometry (hereinafter, also referred to as “ICP Emission Spectrometry”).

(Method of Producing Catalyst (D))

A method of producing the catalyst (D) used for the method of producing C2 to C4 olefin of the present embodiment will be described.

The method of producing the catalyst (D) is not particularly limited, but the method preferably includes:

(i) a process of preparing a solution or a dispersion liquid of transition metal salts;

(ii) a process of generating a precipitate by mixing a precipitant with the solution or the dispersion liquid prepared from the process (i), to obtain a suspension;

(iii) a process of separating the precipitate from the suspension obtained from the process (ii), washing the obtained precipitate, and drying the precipitate to obtain a dry matter;

(iv) a process of impregnating the dry matter obtained from the process (iii) with alkali metal salts or alkali earth metal salts to obtain an impregnated material; and

(v) a process of performing a heat treatment on the impregnated material obtained from the process (iv) to obtain a catalyst.

However, the process (iv) can be properly omitted when the process is not necessary. Hereinafter, the processes will be specifically described.

<Process (i)>

In the process (i), a solution or a dispersion liquid of transition metal salts is prepared.

Since the obtained catalyst can be easily removed in a purification process, the transition metal salt is desired to be excellent in solubility in water. Examples of the salt include an acetate, a fluoride salt, a chloride salt, a bromide salt, an iodide salt, a carbonate, a sulfate, a nitrate, and hydrates thereof and a metal complex. Among these, since anions can be easily removed by heating, a carbonate and a nitrate are preferred, and a nitrate is more preferred. Examples of the transition metal in the transition metal salt include iron, cobalt, nickel, manganese, copper, zinc, titanium, zirconium, lanthanum, and cerium. Specific examples of the transition metal salt include cobalt nitrate, iron nitrate, nickel nitrate, manganese nitrate, copper nitrate, and zinc nitrate. Among these, a combination of iron nitrate and manganese nitrate is preferred, and the molar ratio thereof, which is represented by “iron nitrate/manganese nitrate”, is preferably 1.22 to 8.95.

The preparation of the solution or the dispersion liquid can be carried out by adding the above-described transition metal salt to a solvent to be dissolved or dispersed therein. Moreover, a mixed solution or a mixed dispersion liquid may be prepared by appropriately mixing the above-described plural kinds of transition metal salts.

The content of the metal ion in the solution or dispersion liquid is preferably in the range of from 3×10⁻⁷% by mass to 20% by mass, more preferably in the range of from 3×10⁻³% by mass to 20% by mass, and still more preferably in the range of from 3×10⁻³% by mass to 20% by mass relative to the mass of the solution or the dispersion liquid. When the content is within the above range, the amount of metal components is not exceedingly small for the production of the catalyst, and the metal components are not aggregated because the amount of the metal components is exceedingly high, therefore, an appropriate catalyst can be produced.

Examples of the solvent to be used, from the viewpoint of the high solubility of an inorganic salt, preferably include a polar solvent such as water, methanol, ethanol, propanol, ethylene glycol, acetonitrile, dimethylformamide, dimethylacetoamide, and N-methylpyrrolidone; more preferably water, methanol, ethanol, propanol, and ethylene glycol; and particularly preferably water.

Further, when a dispersion liquid is prepared, dispersants may be used together in order to improve the dispersibility. Examples of the dispersant include a water-soluble polymer. Specific examples thereof include a polymer having an alkylene ether structure such as polyethylene glycol (PEG) and polypropylene glycol; polyvinyl alcohol; polyvinyl ether; polyacrylate; polyvinylpyrrolidone (PVP); poly(mercaptomethylenestyrene-N-vinyl-2-pyrrolidone); and polyacrylonitrile.

<Process (ii)>

In the process (ii), a precipitate is generated by mixing a precipitant with the solution or the dispersion liquid prepared in the process (i) to obtain a suspension.

Here, the “precipitant” generates a hydroxide ion by being dissolved in a solvent. The precipitant is not particularly limited as long as the precipitant has the above characteristics, but an alkaline compound is preferably used. Examples of the precipitant include sodium hydroxide, potassium hydroxide, ammonia, urea, and ammonium carbonate. Among these, from the viewpoint of easy controlling of the metal composition in the catalyst because metal ions are not included, ammonia, urea, or ammonium carbonate is preferred, and ammonia is more preferred.

The used amount of the precipitant is preferably in the range of from 1 time to 50 times (by mole), more preferably in the range of from 2 times to 30 times (by mole), and still more preferably in the range of from 5 times to 20 times (by mole) relative to the molar quantity of the transition metal salt in the solution or the dispersion liquid obtained from the process (i).

In the process (ii), a suspension is prepared from the solution or the dispersion liquid obtained from the process (i) and for example, the precipitant having the above-described amount. Further, in a precipitant solution, the concentration of the precipitant is preferably in the range of from 0.1% by mass to 50% by mass, more preferably in the range of from 1% by mass to 30% by mass, and still more preferably in the range of from 5% by mass to 25% by mass relative to the mass of the precipitant solution. Subsequently, the precipitant solution is co-flowed with the solution or the dispersion liquid prepared from the process (i) and is added dropwise to a vessel for from 0.1 hours to 10 hours, preferably from 0.5 hour to 5 hours, and more preferably from 1 hour to 3 hours, and after the dropwise addition is completed, the solution is continuously stirred for from 0.5 hours to 8 hours, preferably 0.5 hours to 6 hours, and more preferably 0.5 hours to 4 hours. Subsequently, the solution is preferably left to stand for from 8 hours to 48 hours. In this way, the metal ion contained in the solution or the dispersion liquid obtained from the process (i) is precipitated as a hydroxide, and then a suspension in which the generated hydroxide is suspended can be obtained.

Furthermore, the pH of the suspension is preferably 7 to 14, and more preferably 8 to 14.

<Process (iii)>

In the process (iii), the precipitate (i.e., hydroxide) is separated from the suspension obtained from the process (ii), and then the obtained precipitate is washed and then dried to obtain a dry matter.

After the precipitate is separated from the suspension obtained from the process (ii), a dry matter can be obtained, for example, by filtration, washing the precipitate with water and then drying the precipitate. The drying temperature to obtain the dry matter may be a temperature in which the moisture thereon can be mostly removed, and the temperature thereof is preferably in the range of from 20° C. to 150° C. and more preferably in the range of from 60° C. to 130° C. In addition, the dry time is preferably in the range of from 1 hour to 48 hours, and more preferably in the range of from 12 hours to 36 hours. By satisfying the above conditions, the dry matter having hydroxides, which are generated in the process (ii), as the main component can be obtained.

<Process (iv)>

In the process (iv), an impregnated material is obtained by impregnating the dry matter obtained from the process (iii) with alkali metal salts or alkali earth metal salts. As the method thereof, a generally known method such as an impregnation method or an ion exchange method can be appropriately selected. The particularly preferred method is the impregnation method, and as the impregnation method, an “Incipient Wetness method” is particularly preferred. The Incipient Wetness method is a method of impregnating a porous material with a solution having the same volume as the pore volume of the porous material. That is, when B (g) of a porous material having a pore volume of A (cm³/g) is used, the pore volume becomes A×B (cm³). Therefore, a solution having the same volume as A×B (cm³) is impregnated to the porous material. In addition, a pore volume ratio in a pore size, that is, a pore size distribution can be measured by a general gas absorption method. More specifically, a solution containing alkali metal salts or alkali earth metal salts is prepared with the same volume as the pore volume of the dry matter obtained from the process (iii), and then impregnated to the dry matter obtained from the process (iii). When plural metals are impregnated to the dry matter, a simultaneous impregnation method or a sequential impregnation method can be used, but a simultaneous impregnation method is preferred.

As the alkali metal salt or the alkali earth metal salt, a salt with high solubility on water is preferred, and a carbonate or a nitrate is more preferably used.

Examples of the salt preferably include lithium, sodium, potassium, rubidium, cesium, beryllium, magnesium, calcium, strontium, and barium; more preferably sodium, potassium, rubidium, cesium, magnesium, calcium, strontium, and barium; still more preferably sodium, potassium, magnesium, and calcium; and particularly preferably potassium and magnesium.

The concentration of the alkali metal salt or the alkali earth metal salt in the solution of the alkali metal salt or the solution of the alkali earth metal salt is preferably in the range of from 1% by mass to 70% by mass, and more preferably 5% by mass to 50% by mass relative to the total mass of the solution.

Examples of the solvent used for the solution of the alkali metal salt or the alkali earth metal salt, from the viewpoint of the high solubility of an inorganic salt, preferably include a polar solvent such as water, methanol, ethanol, propanol, ethylene glycol, acetonitrile, dimethylformamide, dimethylacetoamide and N-methylpyrrolidone; more preferably water, methanol, ethanol, propanol and ethylene glycol; and particularly preferably water. These solvents can be used as a mixture of the plural kinds thereof.

The temperature at the time of obtaining the impregnated material is preferably in the range of from 10° C. or more to less than 100° C., more preferably in the range of from 20° C. to 80° C., and still more preferably in the range of from 20° C. to 60° C. In addition, the impregnating time is preferably in the range of from 0.1 hours to 3 hours, more preferably in the range of from 0.5 hours to 2 hours, and still more preferably in the range of from 0.5 hours to 1 hour.

<Process (v)>

In the process (v), a heat treatment is carried out on the impregnated material obtained from the process (iv) to obtain the catalyst (D).

Since hydroxides can be changed to oxides by dewatering, the heating temperature of the impregnated material obtained from the process (iv) is preferably in the range of from 300° C. to 800° C., more preferably in the range of from 300° C. to 600° C., and still more preferably in the range of from 400° C. to 600° C. Further, the heating time is preferably in the range of from 1 hour to 48 hours, more preferably in the range of from 1 hour to 24 hours, and still more preferably in the range of from 1 hour to 12 hours.

In this way, the catalyst (D) having oxides as the main component can be obtained.

The catalyst (D) obtained from the above-described production method can be directly used for the FT reaction, or can be used after performing a treatment such as pulverization, molding, or particle size regulation in advance.

The catalyst (D) can be activated by reduction at a temperature of 200° C. to 500° C. for 1 hour to 24 hours under the hydrogen atmosphere of from the normal pressure to 10 MPa, or under the synthesis gas atmosphere of from the normal pressure to 10 MPa prior to use for the FT reaction. Such an activation treatment is generally carried out in this field, and can be recommended for an efficient activation thereof. Moreover, the molar ratio of hydrogen to carbon monoxide (hereinafter, also referred to as “H₂/CO ratio”) in the synthesis gas in this process, which is represented by “hydrogen/carbon monoxide”, is preferably 0.5 to 5, and more preferably 0.5 to 2.

In the description below, the gas used for the activation treatment is also referred to as “reducing gas” in order to distinguish it from the synthesis gas used for the FT reaction.

The temperature of the activation treatment is preferably in the range of from 250° C. to 450° C., and more preferably in the range of from 280° C. to 430° C.

The pressure of the activation treatment is preferably in the range of from the normal pressure to 10 MPa, and more preferably in the range of from the normal pressure to 3 MPa.

The time for the activation treatment is preferably in the range of from 5 hours to 15 hours, and more preferably in the range of from 8 hours to 12 hours.

In the activation treatment, the ratio (W/F) of the mass of a catalyst (W) (g) relative to the speed of supplying synthesis gas (F) (mol/h) is preferably in the range of from 0.01 g·h/mol to 500 g·h/mol, more preferably in the range of from 1 g·h/mol to 100 g·h/mol, and particularly preferably in the range of from 5 g·h/mol to 30 g·h/mol.

As the reducing gas used for the activation treatment, the hydrogen gas or the synthesis gas can be used. When the synthesis gas is used, the molar ratio of H₂/CO is preferably in the range of from 0.5 to 3.0, more preferably in the range of from 0.5 to 2.5, and still more preferably in the range of from 0.6 to 2.0. In addition, the reducing gas may be the same gas as the synthesis gas used for the reaction.

(Support of Catalyst (D))

The catalyst (D) used for the method of producing C2 to C4 olefin of the present embodiment may be constituted only with the catalyst having the above-described oxides as the main component, or may contain other components such as a carbon support, alumina, silica, titania, zirconia, magnesia, ceria, zinc oxide, a polymer (e.g., polyethylene glycol, polyacrylate, polymethacrylate, polyvinylpyrrolidone, and the like) in addition to the catalyst having oxides as the main component. These components can be used as a support.

Preferred examples of the support component in regard to the catalyst (A) may include a carbon support. Examples of the carbon support include activated carbon, carbon black, a carbon nanofiber, a carbon nanotube, and a fullerene; and preferably activated carbon, carbon black, a carbon nanofiber, and a carbon nanotube; and more preferably activated carbon and carbon black; and particularly preferably activated carbon.

Further, preferred examples of the support component in the catalyst (B) include alumina, silica, titania, zirconia, magnesia, ceria, and zinc oxide. The ratio of the support component may be less than 100% by mass, preferably in the range of from 1% by mass to 99% by mass, more preferably in the range of from 3% by mass to 97% by mass, and still more preferably in the range of from 5% by mass to 95% by mass relative to the total mass of the catalyst (B).

As the catalyst (B), a support having both large pores (a peak pore size of from 30 nm to 300 nm) and small pores (a peak pore size of less than 30 nm) can be used. A pore volume ratio in a certain pore size, that is, a pore size distribution can be determined in accordance with a Barret-Joyner-Halenda (BJH) method (nitrogen is used as a probe) using an automatic absorption measuring apparatus, for example, Autosorb-1 (manufactured by Quantachrome Instruments). Here, a pore size in which the number of pores having the pore size becomes the maximum is referred to as a “peak pore size.” In addition, large pores accelerate dispersion of reaction gas and dispersion of the generated hydrocarbon to the outside of the catalyst, and small pores maintain a high specific surface area and a high dispersion state of a catalyst component. As a result, a catalyst with high activity can be obtained.

The pore volume of a large pore (a peak pore size of from 30 nm to 300 nm) is preferably 30% to 90%, more preferably 50% to 90%, and still more preferably 60% to 90% relative to the entire pore volume. Further, the pore volume of a small pore (a peak pore size of less than 30 nm) is preferably 10% to 70%, more preferably 10% to 50%, and still more preferably 10% to 40% relative to the entire pore volume.

A support having both large pores and small pores described above can be prepared by impregnating a support having only one kind of pore with a dispersing element having nanoparticles or a solution having transition metal salts, and then by carrying out the heat treatment on the obtained impregnated material. The support can be prepared by carrying out the heat treatment on the impregnated material obtained from the treatment using a base such as ammonia, potassium hydroxide, and sodium hydroxide after the solution of the transition metal salt is impregnated to the support. Hereinafter, a support having only one kind of pore is referred to as a “raw material support”, and a support having both large pores and small pores is referred to as a “support having two kinds of pores.”

The kind of the raw material support is not particularly limited, but a pore size thereof is preferably 10 nm to 500 nm, more preferably 30 nm to 400 nm, and particularly preferably 30 nm to 300 nm. Examples of the raw material support include alumina, silica, titania, zirconia, magnesia, ceria, and zinc oxide, and among these, silica is preferred.

The nanoparticle used for preparing a support having two kinds of pores is not particularly limited as long as the nanoparticle can be supported in the pores of the raw material support, but the dispersion particle size thereof which can be obtained by a dynamic light scattering method is preferably 0.1 nm to 50 nm, more preferably 0.1 nm to 30 nm, and particularly preferably 5 nm to 25 nm. Examples of the nanoparticle include oxides of aluminum, silicon, titanium, zirconium, magnesium, cerium, manganese and zinc, a complex oxide, a hydroxide, and a complex hydroxide, and preferred examples thereof include silica and zirconia. A dispersing element containing these nanoparticles may be used in a mixture of the plural kinds thereof.

Since the transition metal can be easily removed from the obtained catalyst in the purification process, a salt with excellent solubility on water is preferred as the transition metal salt used for the preparation of the support having two kinds of pores. Examples of such a salt include an acetate, a fluoride salt, a chloride salt, a bromide salt, an iodide salt, a carbonate, a sulfate, a nitrate, an oxychloride salt, oxynitrate, and hydrates thereof and a transition metal complex. Among these, since anions can be easily removed by heating, a nitrate is preferably used. Examples of the transition metal in the transition metal salt include iron, cobalt, nickel, manganese, copper, zinc, titanium, zirconium, lanthanum and cerium.

The preparation of the solution of the transition metal salt can be carried out by adding the above-described transition metal salt to a solvent to be dissolved therein. Moreover, a mixed solution or a mixed dispersion liquid may be prepared by appropriately mixing the above-described plural kinds of transition metal salts.

Examples of the solvent to be used, from the viewpoint of high solubility of an inorganic salt, preferably include a polar solvent such as water, methanol, ethanol, propanol, ethylene glycol, acetonitrile, dimethylformamide, dimethylacetoamide, and N-methylpyrrolidone; more preferably water, methanol, ethanol, propanol, and ethylene glycol; and particularly preferably water. These solvents may be used in a mixture of the plural kinds thereof.

In the preparation of a support having two kinds of pores, the temperature during the heat treatment on the impregnated material obtained by impregnating the support having only one kind of pore with the dispersing element having nanoparticles or the solution having the transition metal salt is preferably in the range of from 200° C. to 800° C., and more preferably 300° C. to 700° C. In addition, the heating time is preferably in the range of from 1 hour to 48 hours, and more preferably 1 hour to 10 hours.

In this way, small pores are formed, and therefore a support having two kinds of pores can be obtained.

When the catalyst (D) contains a support component, the content ratio of catalyst metal in the catalyst (D) (here, the catalyst metal represents metal that does not correspond to the support component in the catalyst (D) containing the support component) is not particularly limited as long as the catalyst used for the production reaction of the light olefin of the present embodiment has a ratio in which the smooth catalytic activity can be obtained. The ratio of the catalyst metal in the catalyst (D) may be less than 100% by mass, preferably in the range of from 1% by mass to 99% by mass, more preferably in the range of 3% by mass to 97% by mass, and still more preferably in the range of 5% by mass to 95% by mass relative to the total mass of the catalyst (D).

When the above-described support component is introduced to a catalyst, an appropriate method therefor can be selected from known methods, which are generally used, such as a precipitation method, a gelation method, an impregnation method, and an ion exchange method.

A particularly preferred method as the method of introducing a support component to the catalyst (A) is a method of dispersing a support component in the solution or the dispersion liquid in the process (i), and precipitating the support component together with the precipitate generated by adding the precipitant thereto in the process (ii). With respect to the amount of the support component added to the solution or the dispersion liquid in the process (i), the ratio of the catalyst metal is preferably in the range of from 1% by mass to 99% by mass, more preferably in the range of from 3% by mass to 97% by mass, and still more preferably in the range of from 5% by mass to 95% by mass relative to the total mass of the catalyst (A). That is, the amount of the support component is preferably in the range of from 1% by mass to 99% by mass, more preferably in the range of from 3% by mass to 97% by mass, and still more preferably in the range of from 5% by mass to 95% by mass relative to the total mass of the catalyst (A).

A particularly preferred method as a method of introducing a support component to the catalyst (B) is a method of introducing a cobalt salt solution to the catalyst in accordance with the impregnation method, and carrying out the heat treatment on the impregnated material. At this time, a solution containing manganese and zinc as a promotor can be simultaneously or sequentially impregnated to the catalyst.

Examples of the cobalt salt to be used include a nitrate, an acetate, a carbonate and a sulfate, among these, a nitrate is preferred.

The preparation of a solution of the cobalt salt can be carried out by adding the above-described cobalt salt to a solvent and dissolving the cobalt salt therein.

Examples of the solvent to be used, from the viewpoint of high solubility of an inorganic salt, preferably include a polar solvent such as water, methanol, ethanol, propanol, ethylene glycol, acetonitrile, dimethylformamide, dimethylacetoamide and N-methylpyrrolidone; more preferably water, methanol, ethanol, propanol and ethylene glycol; and particularly preferably water. These solvents may be used in a mixture of the plural kinds thereof.

Various known methods can be used as the impregnation method, but the Incipient Wetness method is preferably used.

Subsequently, the obtained impregnated material is subjected to the heat treatment. The heating temperature is preferably in the range of from 300° C. to 800° C., and the heating time thereof is preferably in the range of from 1 hour to 48 hours.

In this way, the catalyst used for the FT reaction can be prepared.

(Synthesis Gas)

As the synthesis gas used for producing C2 to C4 olefin of the present embodiment, gas containing hydrogen and carbon monoxide, gas containing hydrogen and carbon dioxide, and gas containing hydrogen, carbon monoxide, and carbon dioxide can be used. Among these, particularly, the total volume of hydrogen and carbon monoxide is preferably from 50% by volume to 100% by volume relative to the total volume of the synthesis gas. When such synthesis gas is used, the productivity thereof becomes high. Since the hydrogenation reaction of the carbon monoxide is smoothly carried out and the productivity becomes high, the molar ratio of the hydrogen to the carbon monoxide in the synthesis gas, which is represented by “hydrogen/carbon monoxide”, is preferably 0.3 or more. Further, the molar ratio of the hydrogen to the carbon monoxide in the synthesis gas is preferably 3 or less in order to prevent the degradation of the productivity due to the exceedingly low existing amount of the carbon monoxide in the raw material gas.

The molar ratio of the hydrogen to the carbon monoxide in the synthesis gas, which is represented by “hydrogen/carbon monoxide”, is more preferably in the range of from 0.5 to 3.0, still more preferably in the range of from 0.5 to 2.5, and particularly preferably in the range of from 0.6 to 2.0.

(FT Reaction)

The FT reaction of the present invention uses a dispersion medium, and the number of carbon atoms of the hydrocarbon product is barely increased due to the effects of an immediate extraction of the hydrocarbon product from a catalyst by the dispersion medium, and therefore it is estimated that the hydrocarbon product with the high content of olefin having 2 to 4 carbon atoms can be obtained. The FT reaction is desired to allow the above-described synthesis gas and the above-described catalyst to continuously react with each other using a slurry bed liquid-phase synthesis process. The pressure of the FT reaction is preferably in the range of from 0.1 MPa to 30 MPa, more preferably in the range of from 0.1 MPa to 10 MPa, and particularly preferably in the range of from 0.5 MPa to 3 MPa. Here, “the pressure of the FT reaction” represents the pressure inside a reaction vessel.

The above-described catalyst is preferably dispersed in a dispersion medium in the reaction vessel in advance, for example, to become a slurry state. As the dispersion medium, it is preferable that an organic compound which becomes a liquid state at a reaction temperature and under a reaction pressure in the process of reacting the synthesis gas with the catalyst (D). As the dispersion medium, for example, an organic compound which becomes a liquid state in the temperature range of from 100° C. to 600° C. under the normal pressure can be used. Here, the “organic compound which becomes a liquid state in the temperature range of from a° C. to b° C.” means an organic compound which becomes a liquid state at a temperature of a° C. or a temperature of b° C. among the range of from a° C. to b° C. The “normal pressure” means 0.1 MPa. As the dispersion medium, it is preferable that an organic compound which becomes a liquid state in the temperature range from 150° C. to 400° C. is used, it is more preferable that an organic compound which becomes a liquid state in the temperature range from 150° C. to 350° C. is used, it is still more preferable that an organic compound which becomes a liquid state in the temperature range from 200° C. to 330° C. is used, and it is particularly preferable that an organic compound which becomes a liquid state in the temperature range from 200° C. to 300° C. is used. Such an organic compound can be preferably used as a dispersion medium under the FT reaction condition. Examples of the organic compound include a hydrocarbon compound and an oxygen-containing hydrocarbon compound. Examples of the hydrocarbon compound preferably include paraffin having about 10 to 100 carbon atoms such as decane, undecane, dodecane, tridecane, tetradecane, pentadecane, hexadecane, heptadecane, octadecane, nonadecane, and eicosane and the mixtures thereof; and paraffin having about 10 to 100 carbon atoms as a by-product from the FT reaction (generally referred to as “FT wax”) or commercially available polyalphaolefin having about 10 to 100 carbon atoms can be used as well. Examples of the oxygen-containing hydrocarbon compound preferably include alcohol having about 10 to 100 carbon atoms such as decanol, undecanol, dodecanol, tridecanol, tetradecanol, pentadecanol, hexadecanol, heptadecanol, octadecanol, nonadecanol, and eicosadecanol; a carboxylic acid having about 10 to 100 carbon atoms such as a decanoic acid, an undecanoic acid, a dodecanoic acid, a tridecanoic acid, a tetradecanoic acid, a pentadecanoic acid, a hexadecanoic acid, a heptadecanoic acid, an octadecanoic acid, a nonadecanoic acid, and an eicosanic acid; polyethylene glycol; polypropylene glycol; silicone, and the mixtures thereof. Further, a hydrocarbon compound is preferably used as the organic compound.

The ratio of the catalyst (D) to the dispersion medium is basically optional, but the ratio of the dispersion medium per 1 g of the catalyst (D) is preferably in the range of from 1 mL to 10 L, more preferably in the range of from 5 mL to 2 L, and still more preferably in the range of from 10 mL to 1 L.

The reaction temperature of the FT reaction in the method of producing C2 to C4 olefin of the present embodiment is preferably in the range of from 100° C. to 600° C., more preferably in the range of from 200° C. to 500° C., still more preferably in the range of from 250° C. to 400° C., and particularly preferably in the range of from 250° C. to 350° C.

Furthermore, with regard to the FT reaction in the method of producing C2 to C4 olefin of the present embodiment, the ratio (W/F) of the mass of the catalyst (W) (g) to the supply speed (F) (mol/h) per mole of the synthesis gas is preferably in the range of from 0.01 g·h/mol to 100 g·h/mol, more preferably in the range of from 1.0 g·h/mol to 50 g·h/mol, and particularly preferably in the range of from 5.0 g·h/mol to 30 g·h/mol.

The FT reaction time, in the case of continuous reaction, is represented by the ratio (V/F′) of the reaction volume (V) (mL) to the supply speed (F′) (mL/h) per volume of the synthesis gas, and the ratio thereof is preferably in the range of from 1.0×10⁻⁵ h to 50 h, more preferably in the range of from 1.0×10⁻³ h to 20 h, and still more preferably in the range of from 4.0×10⁻³ h to 5 h.

The product generated from the FT reaction can be obtained as a mixture of a plurality of compounds (i.e., hydrocarbon), the abundance ratio of respective compounds in the product can be analyzed using a known gas chromatography technique. In this way, the compositions of the components of respective hydrocarbons obtained from the FT reaction can be calculated.

A hydrocarbon product with the high content of olefin having 2 to 4 carbon atoms can be obtained from the above-described production method. In regard to the content of olefin having 2 to 4 carbon atoms, the ratio of the total carbon atoms constituting olefin having 2 to 4 carbon atoms relative to the total carbon atoms constituting the hydrocarbon product obtained from the above-described production method is preferably in the range of from 18% to 100%, more preferably in the range of from 24% to 100%, still more preferably in the range of from 30% to 100%, particularly preferably in the range of from 35% to 100%, and further still more preferably in the range of from 40% to 100%.

According to the production method described above, the content of olefin having 2 to 4 carbon atoms among the product, particularly the content of propylene can be enhanced.

Further, in the present embodiment, a process of catalytically cracking the product obtained from the process of reacting the synthesis gas with the catalyst (D) can be carried out after the reaction process of the synthesis gas with the catalyst (D). The process of catalytically cracking the product includes the same process as the second process in a second embodiment to be described below.

### Second Embodiment

The method of producing olefin having 2 to 4 carbon atoms of the present embodiment include a first process of reacting synthesis gas and a catalyst (E) in the presence of a dispersion medium to produce a hydrocarbon product through a Fischer-Tropsch reaction, and a second process of catalytically cracking the hydrocarbon produced by allowing the hydrocarbon product to come into contact with a cracking catalyst which is consisting of zeolite containing one or more kinds of elements selected from the group consisting of alkali metal, alkali earth metal and transition metal.

In this way, the content of olefin having 2 to 4 carbon atoms, particularly the content of propylene can be further enhanced.

<First Process>

The first process is desired to include a process of reacting at least one kind of a catalyst (E) selected from the group consisting of below catalysts (A) to (C) with synthesis gas in the presence of a dispersion medium through the Fischer-Tropsch reaction.

Catalyst (A): a catalyst containing iron;

Catalyst (B): a catalyst containing cobalt; and

Catalyst (C): a catalyst containing nickel or ruthenium

Here, the catalyst (B) contains cobalt, provided that the catalyst (B) may be a catalyst which excludes a catalyst obtained by reducing a cobalt ion and an iron ion in a dispersion liquid or a solution containing the cobalt ion, the iron ion and a dispersant that interacts with the cobalt ion and the iron ion, and examples of such a catalyst (B) include the same catalysts as those described in the first embodiment.

(Catalyst (E))

Further, the catalyst (A) may further contain one or more metal elements selected from the group consisting of cobalt, nickel and ruthenium.

Furthermore, the catalyst (B) may further contain one or more metal elements selected from the group consisting of iron, alkali metal, alkali earth metal, nickel and ruthenium.

Furthermore, the catalyst (C) may further contain one or more metal elements selected from the group consisting of iron, alkali metal, alkali earth metal, and cobalt.

Furthermore, the catalysts (A) to (C) may be used in combination.

The catalysts (A) to (C) may contain one to three kinds of other transition metal elements as a promotor. The transition metal element is preferably manganese, copper, zinc, titanium, zirconium lanthanum, or cerium, more preferably manganese or copper, and particularly preferably manganese. When the catalyst (A) contains the transition metal element as a promotor, the content of iron is preferably 50 mole % to 90 mole %, the total content of alkali metal and alkali earth metal is preferably 0.5 mole % to 10 mole %, and the total content of the transition metal element as a promotor is preferably 9.5 mole % to 48 mole %, relative to the total number of moles in the iron, the alkali metal, the alkali earth metal and the transition metal element as a promotor. More preferably, the content of iron is 50 mole % to 90 mole %, the total content of alkali metal and alkali earth metal is 0.5 mole % to 10 mole %, and the total content of the transition metal element as a promotor is 9.5 mole % to 45 mole %.

When the catalyst (B) contains the transition metal element as a promotor, the mass ratio of the transition metal element as a promotor relative to cobalt, which is represented by “the total of the transition metal element as a catalyst/cobalt”, is preferably in the range of from 0.01 to 5.

When the catalyst (C) contains the transition metal element as a promotor, the mass ratio of the transition metal element as a promotor to nickel or ruthenium, which is represented by “the total of the transition metal element as a promotor/nickel or ruthenium”, is preferably in the range of from 0.01 to 5.

The catalyst (E) used for the method of producing C2 to C4 olefin of the present embodiment is preferably the catalyst (A) or the catalyst (B), and more preferably the catalyst (B) or the catalyst (A) further containing manganese, that is, the catalyst containing the elements (1) which are iron and manganese and the elements (2) which are one to three kinds of metal elements selected from the group consisting of an alkali metal element and an alkali earth metal element.

The catalyst (A) contains iron, so that the reactivity of the FT reaction can be easily secured, which is preferable.

In addition, the catalyst may contain cobalt or copper other than those described above. When copper is included in the catalyst, the reduction of iron is accelerated in the activation treatment described below, which is preferable.

Examples of the elements (2) preferably include lithium, sodium, potassium, rubidium, cesium, beryllium, magnesium, calcium, strontium and barium; more preferably sodium, potassium, rubidium, cesium, magnesium, calcium, strontium and barium; still more preferably sodium, potassium, magnesium and calcium; and particularly preferably potassium and magnesium.

In addition, when the elements (2) contained in the catalyst (E) are magnesium, a gas shift reaction (i.e., a reaction of generating carbon dioxide and hydrogen by reacting carbon monoxide and water) which is a competition reaction of the FT reaction can be suppressed, which is preferable.

With respect to the molar ratio of the elements (1) to the elements (2) included in the catalyst (E), when the molar ratio of iron is represented by a mole %, the molar ratio of manganese is represented by b mole %, and the total molar ratio of the metal element in the elements (2) is represented by c mole % relative to the total number of moles in the elements of the iron, the manganese, and the metal elements of the elements (2), the molar ratio is preferably 50≦a≦90, 9.5≦b≦45, and 0.5≦c≦10, (provided that a+b+c=100). The selectivity of C2 to C4 olefin is increased by controlling the molar ratio of the catalyst in this range.

The molar ratio of the elements (1) to the elements (2) included in the catalyst (E) is more preferably 55≦a≦85, 9.5≦b≦45, and 1≦c≦7 (provided that a+b+c=100), and still more preferably 60≦a≦80, 15≦b≦40, and 1≦c≦6 (provided that a+b+c=100).

Further, the gas shift reaction can be suppressed by allowing cobalt to be included in the catalyst (B), which is preferable.

The catalyst (B) may include manganese, zinc, or the like in addition to cobalt. When manganese or zinc is included in the catalyst (B), the olefin ratio in a hydrocarbon generated by the FT reaction is increased, which is preferable.

The amount of manganese contained in the catalyst (B) is preferably in the range of from 0.01 times to 5 times (by mass), more preferably in the range of from 0.1 times to 4 times (by mass), and still more preferably in the range of from 0.5 times to 4 times (by mass) relative to the amount of cobalt. Further, the amount of zinc is preferably in the range of from 0.01 times to 5 times (by mass), more preferably in the range of from 0.01 times to 1 time (by mass), and still more preferably in the range of from 0.01 times to 0.2 times (by mass) relative to the content of cobalt.

The catalyst (E) used for the method of producing C2 to C4 olefin of the present embodiment may be a catalyst containing the following elements (3) and (4).

Elements (3): at least one kind of element selected from the group consisting of iron, cobalt and nickel.

Elements (4): one to three kinds of elements selected from the group consisting of alkali metal and alkali earth metal.

The molar ratio of the elements (3) to the elements (4) included in the catalyst (E), which is represented by “the total of the elements (3)/the total of the elements (4)”, is preferably 5 to 180. The reactivity of the FT reaction is easily secured by controlling the molar ratio of the catalyst in this way.

The catalyst (E) used for the method of producing C2 to C4 olefin of the present embodiment is preferably a combination of iron and potassium as catalytic metal, and the molar ratio thereof, which is represented by “iron/potassium”, is preferably 5 to 180. Moreover, the catalyst (E) may further contain manganese, in this case, the content of iron is preferably 50 mole % to 90 mole %, the content of manganese is preferably 9.5 mole % to 48 mole %, and the content of potassium is preferably 0.5 mole % to 10 mole % relative to the total number of moles in the iron, the manganese and the potassium; the content of iron is more preferably 50 mole % to 90 mole %, the content of manganese is more preferably 9.5 mole % to 45 mole %, and the content of potassium is more preferably 0.5 mole % to 10 mole % relative to the total number of moles of the iron, the manganese and the potassium.

Further, the molar ratio of metal contained in the catalyst in the present embodiment may be determined by using Energy Dispersive X-ray Fluorescence Spectrometry (hereinafter, also referred to as “EDS Spectrometry”) or Inductively Coupled Plasma Emission Spectrometry (hereinafter, also referred to as “ICP Emission Spectrometry”).

(Method of Producing Catalyst (E))

A method of producing the catalyst (E) used for the method of producing C2 to C4 olefin of the present embodiment will be described.

The method of producing the catalyst (E) is not particularly limited, but it is preferable that the method preferably includes:

(i) a process of preparing a dispersion liquid or a solution of transition metal salts;

(ii) a process of generating a precipitate by mixing a precipitant with the solution or the dispersion liquid prepared from the process (i) to obtain a suspension;

(iii) a process of separating the precipitate from the suspension obtained from the process (ii), washing the obtained precipitate, and drying the precipitate to obtain a dry matter;

(iv) a process of impregnating the dry matter obtained from the process (iii) with alkali metal salts or alkali earth metal salts to obtain an impregnated material; and (v) a process of performing a heat treatment on the impregnated material obtained from the process (iv) to obtain a catalyst.

However, the process (iv) can be properly omitted when the process is not necessary. Hereinafter, the processes will be specifically described.

<Process (i)>

In the process (i), a solution or a dispersion liquid of transition metal salts is prepared.

Since the transition metal can be easily removed from the obtained catalyst in a purification process, the transition metal salt is desired to be excellent in solubility on water. Examples of the salt include an acetate, a fluoride salt, a chloride salt, a bromide salt, an iodide salt, a carbonate, a sulfate, a nitrate, and hydrates thereof and a metal complex. Among these, since anions can be easily removed by heating, a carbonate or a nitrate is preferred, and a nitrate is more preferred. Examples of the transition metal in the transition metal salt include iron, cobalt, nickel, manganese, copper, zinc, titanium, zirconium, lanthanum and cerium. Specific examples of the transition metal salt include cobalt nitrate, iron nitrate, nickel nitrate, manganese nitrate, copper nitrate and zinc nitrate. Among these, a combination of iron nitrate and manganese nitrate is preferred, and the molar ratio thereof, which is represented by “iron nitrate/manganese nitrate”, is preferably 1.22 to 8.95.

The preparation of the solution or the dispersion liquid can be carried out by adding the above-described transition metal salt to a solvent to be dissolved or dispersed therein. Moreover, a mixed solution or a mixed dispersion liquid may be prepared by appropriately mixing the above-described plural kinds of transition metal salts.

The content of the metal ion in the solution or the dispersion liquid is preferably in the range of from 3×10⁻⁷% by mass to 20% by mass, more preferably in the range of from 3×10⁻⁵% by mass to 20% by mass, and still more preferably in the range of from 3×10⁻³% by mass to 20% by mass relative to the mass of the solution or the dispersion liquid. When the content is within the above range, the number of metal components is not exceedingly small for the production of the catalyst, and the metal components are not aggregated because the number of the metal components is exceedingly high, therefore, an appropriate catalyst can be produced.

Examples of the solvent to be used, from the viewpoint of the high solubility of an inorganic salt, preferably include a polar solvent such as water, methanol, ethanol, propanol, ethylene glycol, acetonitrile, dimethylformamide, dimethylacetoamide and N-methylpyrrolidone; more preferably water, methanol, ethanol, propanol and ethylene glycol; and particularly preferably water.

Further, a dispersant may be used together in order to improve the dispersibility at the time of preparing the dispersion liquid. Examples of the dispersant include a water-soluble polymer. Specific examples thereof include a polymer having an alkylene ether structure such as polyethylene glycol (PEG) and polypropylene glycol; polyvinyl alcohol; polyvinyl ether; polyacrylate; polyvinylpyrrolidone (PVP); poly(mercaptomethylenestyrene-N-vinyl-2-pyrrolidone); and polyacrylonitrile.

<Process (ii)>

In the process (ii), a precipitate is generated by mixing a precipitant with the solution or the dispersion liquid prepared in the process (i) to obtain a suspension.

Here, the “precipitant” generates a hydroxide ion by being dissolved in a solvent. The precipitant is not particularly limited as long as the precipitant has the above characteristics, but an alkaline compound is preferably used. Examples of the precipitant include sodium hydroxide, potassium hydroxide, ammonia, urea and ammonium carbonate. Among these, from the viewpoint of easy controlling of the metal composition in the catalyst because metal ions are not included, ammonia, urea, and ammonium carbonate are preferred, and ammonia is more preferred.

The used amount of the precipitant is preferably in the range of from 1 time to 50 times (by mole), more preferably in the range of from 2 times to 30 times (by mole), and still more preferably in the range of from 5 times to 20 times (by mole) relative to the molar quantity of the transition metal salt in the solution or the dispersion liquid obtained from the process (i).

In the process (ii), a suspension is prepared from with the solution or the dispersion liquid obtained from the process (i) and the precipitant having the above-described amount. Further, in a precipitant solution, the concentration of the precipitant is preferably in the range of from 0.1% by mass to 50% by mass, more preferably in the range of from 1% by mass to 30% by mass, and still more preferably in the range of from 5% by mass to 25% by mass relative to the mass of the precipitant solution. Subsequently, the precipitant solution is co-flowed with the solution or the dispersion liquid prepared from the process (i) and is added dropwise to a vessel for from 0.1 hours to 10 hours, preferably from 0.5 hour to 5 hours, and more preferably from 1 hour to 3 hours. Then the dropwise addition is completed, the solution is continuously stirred for from 0.5 hours to 8 hours, preferably 0.5 hours to 6 hours, and more preferably 0.5 hours to 4 hours. Subsequently, the solution is preferably left to stand for from 8 hours to 48 hours. In this way, the metal ion contained in the solution or the dispersion liquid obtained from the process (i) is precipitated as a hydroxide, and then a suspension in which the generated hydroxide is suspended can be obtained.

Furthermore, the pH of the suspension is preferably 7 to 14, and more preferably 8 to 14.

<Process (iii)>

In the process (iii), the precipitate (i.e., hydroxide) is separated from the suspension obtained from the process (ii), the obtained precipitate is washed and then dried to obtain a dry matter.

After the precipitate is separated from the suspension obtained from process (ii), a dry matter can be obtained, for example, by filtration, washing the precipitate with water and then drying. The drying temperature to obtain the dry matter may be a temperature in which the moisture thereon can be mostly removed, and the temperature thereof is preferably in the range of from 20° C. to 150° C., and more preferably in the range of from 60° C. to 130° C. In addition, the dry time is preferably in the range of from 1 hour to 48 hours, and more preferably in the range of from 12 hours to 36 hours. By satisfying the above conditions, the dry matter having hydroxides, which are generated in the process (ii), as the main component can be obtained.

<Process (iv)>

In the process (iv), an impregnated material is obtained by impregnating the dry matter obtained from the process (iii) with alkali metal salts or alkali earth metal salts. A generally known method such as an impregnation method or an ion exchange method can be appropriately selected. The particularly preferred method is the impregnation method, and as the impregnation method, an “Incipient Wetness method” is particularly preferred. The Incipient Wetness method is a method of impregnating a porous material with a solution having the same volume as the pore volume of the porous material. That is, when B (g) of a porous material having a pore volume of A (cm³/g) is used, the pore volume becomes A×B (cm³). Therefore, a solution having the same volume as A×B (cm³) is impregnated to the porous material. In addition, a pore volume ratio in a pore size, that is, a pore size distribution can be measured by a general gas absorption method. More specifically, a solution containing alkali metal salts or alkali earth metal salts is prepared with the same volume as the pore volume of the dry matter obtained from the process (iii), and then impregnated to the dry matter obtained from the process (iii). When plural metals are impregnated, a simultaneous impregnation method or a sequential impregnation method can be used, but a simultaneous impregnation method is preferred.

As the alkali metal salt or the alkali earth metal salt, a salt with high solubility on water is preferred, and a carbonate and a nitrate are more preferably used.

Examples of the salt preferably include lithium, sodium, potassium, rubidium, cesium, beryllium, magnesium, calcium, strontium and barium; more preferably sodium, potassium, rubidium, cesium, magnesium, calcium, strontium and barium; still more preferably sodium, potassium, magnesium and calcium; and particularly preferably potassium and magnesium.

The concentration of the alkali metal salt or the alkali earth metal salt in the solution of the alkali metal salt or the solution of the alkali earth metal salt is preferably in the range of from 1% by mass to 70% by mass, and more preferably 5% by mass to 50% by mass relative to the total mass of the solution.

Examples of the solvent used for the solution of the alkali metal salt or the alkali earth metal salt, from the viewpoint of the high solubility of an inorganic salt, preferably include a polar solvent such as water, methanol, ethanol, propanol, ethylene glycol, acetonitrile, dimethylformamide, dimethylacetoamide and N-methylpyrrolidone; more preferably water, methanol, ethanol, propanol and ethylene glycol; and particularly preferably water. These solvents can be used as a mixture of the plural kinds thereof.

The temperature to obtain the impregnated material is preferably in the range of from 10° C. or more to less than 100° C., more preferably in the range of from 20° C. to 80° C., and still more preferably in the range of from 20° C. to 60° C. In addition, the impregnating time is preferably in the range of from 0.1 hours to 3 hours, more preferably in the range of from 0.5 hours to 2 hours, and still more preferably in the range of from 0.5 hours to 1 hour.

<Process (v)>

In the process (v), a heat treatment is carried out on the impregnated material obtained from the process (iv) to obtain the catalyst (E).

Since hydroxides can be changed to oxides by dewatering, the heating temperature of the impregnated material obtained from the process (iv) is preferably in the range of from 300° C. to 800° C., more preferably in the range of from 300° C. to 600° C., and still more preferably in the range of from 400° C. to 600° C. Further, the heating time is preferably in the range of from 1 hour to 48 hours, more preferably in the range of from 1 hour to 24 hours, and still more preferably in the range of from 1 hour to 12 hours.

In this way, the catalyst (E) having oxides as the main component can be obtained.

The catalyst (E) obtained from the above-described production method can be directly used for the FT reaction, or can be used after performing a treatment such as pulverization, molding, or particle size regulation in advance.

The catalyst (E) can be activated by reduction at a temperature of 200° C. to 500° C. for 1 hour to 24 hours under the hydrogen atmosphere of from the normal pressure to 10 MPa, or under the synthesis gas atmosphere of from the normal pressure to 10 MPa prior to use for the FT reaction. Such an activation treatment is generally carried out in this field, and can be recommended for an efficient activation thereof. Moreover, the molar ratio of hydrogen to carbon monoxide in the synthesis gas in this process, which is represented by “hydrogen/carbon monoxide”, is preferably 0.5 to 5, and more preferably 0.5 to 2.

In the below description, the gas used for the activation treatment is also referred to as “reducing gas” in order to distinguish it from the synthesis gas used for the FT reaction.

The temperature of the activation treatment is preferably in the range of from 250° C. to 450° C., and more preferably in the range of from 280° C. to 430° C.

The pressure of the activation treatment is preferably in the range of from the normal pressure to 10 MPa, and more preferably in the range of from the normal pressure to 3 MPa.

The time for the activation treatment is preferably in the range of from 5 hours to 15 hours, and more preferably in the range of from 8 hours to 12 hours.

In the activation treatment, the ratio (W/F) of the mass of the catalyst (W) (g) to the speed of supplying the synthesis gas (F) (mol/h) is preferably in the range of from 0.01 g·h/mol to 500 g·h/mol, more preferably in the range of from 1 g·h/mol to 100 g·h/mol, and particularly preferably in the range of from 5 g·h/mol to 30 g·h/mol.

As the reducing gas used for the activation treatment, hydrogen gas or the synthesis gas can be used. When the synthesis gas is used, the molar ratio of H₂/CO is preferably in the range of from 0.5 to 3.0, more preferably in the range of from 0.5 to 2.5, and still more preferably in the range of from 0.6 to 2.0. In addition, the same gas as the synthesis gas used for the reaction with the reducing gas can be used as well.

(Support of Catalyst (E))

The catalyst (E) used for the method of producing C2 to C4 olefin of the present embodiment may be constituted only with the catalyst having the above-described oxides as the main component, or may contain other components such as a carbon support, alumina, silica, titania, zirconia, magnesia, ceria, zinc oxide and a polymer (e.g., polyethylene glycol, polyacrylate, polymethacrylate, polyvinylpyrrolidone, and the like) in addition to the catalyst having oxides as the main component. These components can be used as a support.

Preferred examples of the support component in regard to the catalyst (A) include a carbon support. Examples of the carbon support include activated carbon, carbon black, a carbon nanofiber, a carbon nanotube and a fullerene; and preferably activated carbon, carbon black, a carbon nanofiber and a carbon nanotube; and more preferably activated carbon and carbon black; and particularly preferably activated carbon.

Further, preferred examples of the support component in the catalyst (B) include alumina, silica, titania, zirconia, magnesia, ceria and zinc oxide.

As the catalyst (B), a support having both large pores (a peak pore size of from 30 nm to 300 nm) and small pores (a peak pore size of less than 30 nm) can be used. A pore volume ratio in a certain pore size, that is, a pore size distribution can be determined in accordance with a BJH method (nitrogen is used as a probe) using an automatic absorption measuring apparatus, for example, AUTOSORB-1 (manufactured by Quantachrome Instruments). Here, a pore size in which the number of pores having the above-described pore size becomes the maximum is referred to as a “peak pore size.” In addition, large pores accelerate dispersion of reaction gas and dispersion of the generated hydrocarbon to the outside of the catalyst, and small pores maintain a high specific surface area and a high dispersion state of a catalyst component. As a result, a catalyst with high activity can be obtained.

The pore volume of a large pore (a peak pore size of from 30 nm to 300 nm) is preferably 30% to 90%, more preferably 50% to 90%, and still more preferably 60% to 90% relative to the entire pore volume. Further, the pore volume of a small pore (a peak pore size of less than 30 nm) is preferably 10% to 70%, more preferably 10% to 50%, and still more preferably 10% to 40% relative to the entire pore volume.

A support having both large pores and small pores described above can be prepared by impregnating a support having only one kind of pore size with a dispersing element having nanoparticles or a solution having transition metal salts, and then by carrying out the heat treatment on the obtained impregnated material. The support can be prepared by carrying out the heat treatment on the impregnated material obtained from the treatment using a base such as ammonia, potassium hydroxide and sodium hydroxide after the solution of the transition metal salt is impregnated to the support. Hereinafter, a support having only one kind of pore size is referred to as a “raw material support”, and a support having both large pores and small pores is referred to as a “support having two kinds of pore size.”

The kind of the raw material support is not particularly limited, but a pore size thereof is preferably 10 nm to 500 nm, more preferably 30 nm to 400 nm, and particularly preferably 30 nm to 300 nm. Examples of the raw material support include alumina, silica, titania, zirconia, magnesia, ceria and zinc oxide, and among these, silica is preferred.

The nanoparticle used for preparing the support having two kinds of pores is not particularly limited as long as the nanoparticle can be supported in the pores of the raw material support, but the dispersion particle size thereof which can be obtained by a dynamic light scattering method is preferably 0.1 nm to 50 nm, more preferably 1 nm to 30 nm, and particularly preferably 5 nm to 25 nm. Examples of the nanoparticle include oxides of aluminum, silicon, titanium, zirconium, magnesium, cerium, manganese and zinc, a complex oxide, a hydroxide and a complex hydroxide, and preferred examples thereof include silica and zirconia. A dispersing element containing these nanoparticles may be used in a mixture of the plural kinds thereof.

Since the transition metal can be easily removed from the obtained catalyst in the purification process, a salt with excellent solubility on water is preferred as the metal salt used for the preparation of the support having two kinds of pores. Examples of such a salt include an acetate, a fluoride salt, a chloride salt, a bromide salt, an iodide salt, a sulfate, a nitrate, an oxychloride salt, oxynitrate, and hydrates thereof and a metal complex. Among these, since anions can be easily removed by heating, a nitrate is preferably used. Examples of the transition metal in the transition metal salt include iron, cobalt, nickel, manganese, copper, zinc, titanium, zirconium, lanthanum and cerium.

The preparation of the solution of the transition metal salt can be carried out by adding the above-described transition metal salt to a solvent to be dissolved. Moreover, a mixed solution or a mixed dispersion liquid may be prepared by appropriately mixing the above-described plural kinds of transition metal salts.

Examples of the solvent to be used, from the viewpoint of high solubility of an inorganic salt, preferably include a polar solvent such as water, methanol, ethanol, propanol, ethylene glycol, acetonitrile, dimethylformamide, dimethylacetoamide and N-methylpyrrolidone; more preferably water, methanol, ethanol, propanol and ethylene glycol; and particularly preferably water. These solvents may be used in a mixture of the plural kinds thereof.

In the preparation of the support having two kinds of pores, the temperature during the heat treatment is preferably in the range of from 200° C. 800° C., and more preferably 300° C. to 700° C. In addition, the heating temperature is preferably in the range of from 1 hour to 48 hours, and more preferably 1 hour to 10 hours.

In this way, the support having two kinds of pores can be obtained.

When the catalyst (E) contains a support component, the content ratio of catalyst metal in the catalyst (E) (here, the catalyst metal represents the metal that does not correspond to the support component in the catalyst (E) containing the support component) is not particularly limited as long as the catalyst used for the production reaction of the light olefin of the present embodiment has a ratio in which the smooth catalytic activity can be obtained. The ratio of the catalyst metal in the catalyst (E) is less than 100% by mass, preferably in the range of from 1% by mass to 99% by mass, more preferably in the range of 3% by mass to 97% by mass, and still more preferably in the range of 5% by mass to 95% by mass relative to the total mass of the catalyst. The amount of the support component is preferably in the range of from 1% by mass to 99% by mass, more preferably in the range of from 3% by mass to 97% by mass, and still more preferably in the range of from 5% by mass to 95% by mass relative to the total mass of the catalyst.

When the above-described support component is introduced to a catalyst, an appropriate method therefor can be selected from known methods, which are generally used, such as a precipitation method, a gelation method, an impregnation method, and an ion exchange method.

A particularly preferred method as the method of introducing a support component to the catalyst (A) is a method of dispersing a support component in the solution or the dispersion liquid in the process (i), and precipitating the support component together with the precipitate generated by adding the precipitant thereto in the process (ii). With respect to the amount of the support component added to the solution or the dispersion liquid in the process (i), the ratio of the catalyst metal in the catalyst is preferably in the range of from 1% by mass to 99% by mass, more preferably in the range of from 3% by mass to 97% by mass, and still more preferably in the range of from 5% by mass to 95% by mass relative to the total mass of the catalyst (A).

A particularly preferred method as a method of introducing a support component to the catalyst (B) is a method of introducing a cobalt salt solution to the catalyst in accordance with the impregnation method, and carrying out the heat treatment on the impregnated material. At this time, a solution containing manganese and zinc as a promotor can be simultaneously or sequentially impregnated.

Examples of the cobalt salt to be used include a nitrate, an acetate, a carbonate and a sulfate, among these, a nitrate is preferred.

The preparation of a solution of the cobalt salt can be carried out by adding the above-described cobalt salt to a solvent and dissolving the cobalt salt therein.

Examples of the solvent to be used, from the viewpoint of high solubility of an inorganic salt, preferably include a polar solvent such as water, methanol, ethanol, propanol, ethylene glycol, acetonitrile, dimethylformamide, dimethylacetoamide and N-methylpyrrolidone; more preferably water, methanol, ethanol, propanol and ethylene glycol; and particularly preferably water. These solvents may be used in a mixture of the plural kinds thereof.

Various known methods can be used as the impregnation method, but the Incipient Wetness method is preferably used.

Subsequently, the obtained impregnated material is subjected to the heat treatment. The heating temperature is preferably in the range of from 300° C. to 800° C., and the heating time thereof is preferably in the range of from 1 hour to 48 hours.

In this way, the catalyst used for the FT reaction can be prepared.

(Synthesis Gas)

As the synthesis gas used for producing C2 to C4 olefin of the present embodiment, gas containing hydrogen and carbon monoxide, gas containing hydrogen and carbon dioxide, and gas containing hydrogen, carbon monoxide, and carbon dioxide can be used. Among these, particularly, the total volume of hydrogen and carbon monoxide is preferably 50% by volume to 100% by volume relative to the total volume of the synthesis gas. When such synthesis gas is used, the productivity thereof becomes high. Since the hydrogenation reaction of the carbon monoxide is smoothly carried out and the productivity becomes high, the molar ratio of the hydrogen to the carbon monoxide in the synthesis gas, which is represented by “hydrogen/carbon monoxide”, is preferably 0.3 or more. Further, the molar ratio of the hydrogen to the carbon monoxide in the synthesis gas is preferably 3 or less in order to prevent the degradation of the productivity due to the exceedingly low existing amount of the carbon monoxide in the raw material gas.

The molar ratio of the hydrogen to the carbon monoxide in the synthesis gas, which is represented by “hydrogen/carbon monoxide”, is more preferably in the range of from 0.5 to 3.0, still more preferably in the range of from 0.5 to 2.5, and particularly preferably in the range of from 0.6 to 2.0.

(FT Reaction)

The FT reaction of the present invention uses a dispersion medium, and the number of carbon atoms of the hydrocarbon product is barely increased due to the effects of an immediate extraction of the hydrocarbon product from a catalyst by the dispersion medium, and therefore it is estimated that the hydrocarbon product with the high content of olefin having 2 to 4 carbon atoms can be obtained. The FT reaction is desired to allow the above-described synthesis gas and the above-described catalyst to continuously react with each other using a slurry bed liquid-phase synthesis process. Further, the pressure of the FT reaction is preferably in the range of from 0.1 MPa to 30 MPa, more preferably in the range of from 0.1 MPa to 10 MPa, and particularly preferably in the range of from 0.5 MPa to 3 MPa. Here, “the pressure of the FT reaction” represents the pressure inside a reaction vessel.

The above-described catalyst is preferably dispersed in a dispersion medium in the reaction vessel in advance, for example, to become a slurry state. As the dispersion medium, it is preferable that an organic compound which becomes a liquid state at a reaction temperature and under a reaction pressure in the process of reacting the synthesis gas with the catalyst (D). As the dispersion medium, for example, an organic compound which becomes a liquid state in the temperature range of from 100° C. to 600° C. under the normal pressure can be used. Here, the “organic compound which becomes a liquid state in the temperature range of from a° C. to b° C.” means an organic compound which becomes a liquid state at a temperature of a° C. or a temperature of b° C. among the range of from a° C. to b° C. The “normal pressure” means 0.1 MPa. As the dispersion medium, it is preferable that an organic compound which becomes a liquid state in the temperature range from 150° C. to 400° C. is used, it is more preferable that an organic compound which becomes a liquid state in the temperature range from 150° C. to 350° C. is used, it is still more preferable that an organic compound which becomes a liquid state in the temperature range from 200° C. to 330° C. is used, and it is particularly preferable that an organic compound which becomes a liquid state in the temperature range from 200° C. to 300° C. is used. Such an organic compound can be preferably used as a dispersion medium under the FT reaction condition. Examples of the organic compound include a hydrocarbon compound and an oxygen-containing hydrocarbon compound. Examples of the hydrocarbon compound preferably include paraffin having about 10 to 100 carbon atoms such as decane, undecane, dodecane, tridecane, tetradecane, pentadecane, hexadecane, heptadecane, octadecane, nonadecane, and eicosane, and mixtures thereof, and paraffin (generally referred to as “FT wax”) having about 10 to 100 carbon atoms as a by-product from the FT reaction or commercially available polyalphaolefin having about 10 to 100 carbon atoms can be used as well. Examples of the oxygen-containing hydrocarbon compound preferably include alcohol having about 10 to 100 carbon atoms such as decanol, undecanol, dodecanol, tridecanol, tetradecanol, pentadecanol, hexadecanol, heptadecanol, octadecanol, nonadecanol, and eicosadecanol; a carboxylic acid having about 10 to 100 carbon atoms such as a decanoic acid, an undecanoic acid, a dodecanoic acid, a tridecanoic acid, a tetradecanoic acid, a pentadecanoic acid, a hexadecanoic acid, a heptadecanoic acid, an octadecanoic acid, a nonadecanoic acid, and an eicosanic acid; polyethylene glycol; polypropylene glycol; silicone, and mixtures thereof. Further, a hydrocarbon compound is preferably used as the organic compound.

The ratio of the catalyst (E) to the dispersion medium is basically optional, but the ratio of the dispersion medium per 1 g of the catalyst (E) is preferably in the range of from 1 mL to 10 L, more preferably in the range of from 5 mL, to 2 L, and still more preferably in the range of from 10 mL to 1 L.

The reaction temperature of the FT reaction in the method of producing C2 to C4 olefin of the present embodiment is preferably in the range of from 100° C. to 600° C., more preferably in the range of from 200° C. to 500° C., still more preferably in the range of from 250° C. to 400° C., and particularly preferably in the range of from 250° C. to 350° C.

Furthermore, with regard to the FT reaction in the method of producing C2 to C4 olefin of the present embodiment, the ratio (W/F) of the mass of the catalyst (W) (g) relative to the supply speed (F) (mol/h) per mole of the synthesis gas is preferably in the range of from 0.01 g·h/mol to 100 g·h/mol, more preferably in the range of from 1.0 g·h/mol to 50 g·h/mol, and particularly preferably in the range of from 5.0 g·h/mol to 30 g-h/mol.

In regard to the FT reaction time, in the case of continuous reaction, the reaction volume (V) (mL) is represented by the ratio (V/F′) of the supply speed (F′) (mL/h) per volume of synthesis gas, and the ratio thereof is preferably in the range of from 1.0×10⁻⁵ h to 50 h, more preferably in the range of from 1.0×10⁻³ h to 20 h, and still more preferably in the range of from 4.0×10⁻³ h to 5 h.

The product generated from the FT reaction can be obtained as a mixture of a plurality of compounds (i.e., hydrocarbon), and the abundance ratio of respective compounds in the product can be analyzed using a known gas chromatography technique. In this way, the compositions of the components of respective hydrocarbons obtained from the FT reaction can be calculated.

The hydrocarbon compound produced by the FT reaction of the first process preferably includes olefin having 2 to 4 carbon atoms, of which the ratio of the total carbon atoms is in the range of more than 20 mole % carbon to 100 mole % carbon, more preferably in the range of from 50 mole % carbon to 100 mole % carbon, and still more preferably in the range of from 60 mole % carbon to 100 mole % carbon. That is, in regard to the hydrocarbon compound produced in the first process, the total amount of olefin combining olefin having 2 to 4 carbon atoms with olefin having 5 or more carbon atoms is preferably in the range of more than 20 mole % carbon to 100 mole % carbon, more preferably in the range of from 50 mole % carbon to 100 mole % carbon, and still more preferably in the range of from 60 mole % carbon to 100 mole % carbon.

Further, the term “mole % carbon” represents “a ratio of total carbon atoms constituting olefin relative to total carbon atoms constituting a hydrocarbon product to be obtained.”

In regard to catalytic cracking of the second process to be described later, for example, in the case where hexane which is paraffin (saturated hydrocarbon) is used as a starting material, two molecules of one molecule of paraffin (propane) and one molecule of olefin (propylene) can be obtained from one molecule of paraffin as shown in the below chemical formula 1. On the other hand, in the case where hexene which is olefin is used as a starting material as shown in the below chemical formula 2, two molecules of olefin (propylene) can be generated from one molecule of olefin, therefore, it is possible to obtain olefin with high efficiency.

[Chem. 1]

C₆H₁₄→H₂C=CHCH₃+CH₃CH₂CH₃ (1)

[Chem. 2]

C₆H₁₂→2H₂C=CHCH₃ (2)

Accordingly, in the case where a hydrocarbon compound containing olefin with the above-described ratio can be obtained from the first process, the content of olefin having 2 to 4 carbon atoms, particularly the content of propylene can be further improved by the catalytic cracking carried out in the second process.

The hydrocarbon compound produced from the FT reaction of the first process preferably contains propylene within the range of from 3 mole % carbon to 100 mole % carbon, more preferably in the range of from 5 mole % carbon to 100 mole % carbon, and still more preferably in the range of from 10 mole % carbon to 100 mole % carbon.

Accordingly, in the first process of the present embodiment, the conditions of reaction such as the kind of a catalyst or the temperature condition during the FT reaction can be selected properly such that a hydrocarbon compound containing olefin with the above-described ratio can be obtained.

<Second Process>

Next, the second process will be described. The second process includes a process of catalytically cracking the hydrocarbon product obtained from the first process in the presence of a cracking catalyst. By including the process of catalytically cracking as the second process, the content of olefin having 2 to 4 carbon atoms, particularly the content of propylene can be further improved.

In regard to the catalytic cracking process, known reactors, which are generally used, can be used as a reactor for performing the catalytic cracking. Examples thereof include a fixed bed reactor, a moving bed reactor, and a fluidized bed reactor.

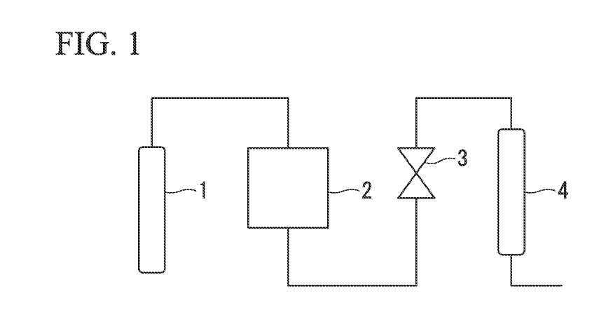

FIG. 1 is a diagram illustrating an example of a production apparatus for conducting the method of producing olefin having 2 to 4 carbon atoms of the present embodiment, and is a schematic diagram illustrating a production apparatus of olefin having 2 to 4 carbon atoms including the first process (i.e., FT reaction) and the second process (i.e., catalytic cracking reaction).

The production apparatus of olefin having 2 to 4 carbon atoms shown in FIG. 1 includes a tank 1 housing synthesis gas, a first reactor 2 carrying out the first process using the synthesis gas supplied from the tank 1, and a second reactor 4 carrying out the catalytic cracking using a reactant obtained by the first reactor 2. The tank 1, the first reactor 2, and the second reactor 4 are connected with each other in this order. In addition, a back pressure valve 3 is provided between the first reactor 2 and the second reactor 4, and controls the pressure of respective reactors of the first reactor 2 and the second reactor 4.

In addition, as production equipment of olefin having 2 to 4 carbon atoms, a cold trap for capturing a liquid product may be appropriately provided.

In the catalytic cracking, a cracking catalyst consisting of zeolite containing one or more kinds of elements selected from the group composed alkali metal, alkali earth metal and transition metal is used.

As the zeolite, when the above-described metal is introduced thereto, either of natural zeolite or synthetic zeolite can be used, but preferably zeolite socony mobil-5 (ZSM-5) type is used. In regard to ZSM-5, the molar ratio of SiO₂ to Al₂O₃, which is represented by “SiO₂/Al₂O₃”, is preferably in the range of from 50 to 4000 (the molar ratio of Si relative to Al (hereinafter, also referred to as the “Si/Al ratio”), which is represented by “Si/Al”, is in the range of from 25 to 2000), more preferably in the range of from 90 to 1000 (the Si/Al ratio is in the range of from 45 to 500), and particularly preferably in the range of from 200 to 800 (the Si/Al ratio is in the range of from 100 to 400).

In addition, the acid property and durability such as acid strength and density of the cracking catalyst can be improved by treating the cracking catalyst by a phosphorous-containing compound, a lanthanum-containing compound, an alkali earth metal-containing compound or the like.

Further, the general definition of zeolite is “crystalline porous alminosilicate and metallosilicate.” A unit cell composition of the ZSM-5 is represented by Mₙ[AlₙSi₉₆₋ₙO₁₉₂].xH₂O. M represents a cation such as a proton, an ammonium cation or a metal cation, n represents a number of more than 0 and less than 27, and x represents a number of more than 0. Hereinafter, the ZSM-5 of which M is a proton is particularly called HZSM-5 in some cases.

The cracking catalyst is preferably zeolite containing one or more kinds of elements selected from the group consisting of alkali metal, alkali earth metal and a d-block element, and the “d-block element” represents an element from among the group 3 element to the group 12 element in the periodic table except lanthanoid and actinoid. Further, the total mass of metal elements introduced to the zeolite is preferably in the range of from 0.01% by mass to 30% by mass, more preferably in the range of from 0.05% by mass to 20% by mass, and particularly preferably in the range of from 0.1% by mass to 10% by mass, relative to the total mass of the cracking catalyst (i.e., the mass after allowing the metal to be introduced in zeolite).

Further, these metal elements are preferably introduced to zeolite through metal-oxygen bonding. Specific examples thereof include a Ba—O bonding, a Mn—O bonding, and a Cu—O bonding. Among these, a Ba—O bonding is preferred. The content ratio (molar ratio) of the metal element, which is represented by “SiO₂: Al₂O₃: oxides of metal elements”, is preferably 50 to 4000:1:0.1 to 50. More specifically, the content ratio (molar ratio) of barium, which is represented by “SiO₂:Al₂O₃:BaO”, is preferably 50 to 4000:1:0.1 to 50.

As the alkali metal contained in the above zeolite, lithium, sodium, potassium, rubidium or cerium is preferred.

Examples of the alkali earth metal contained in the above zeolite preferably include beryllium, magnesium, calcium, strontium, and barium, and more preferably magnesium, calcium, strontium, and barium, still more preferably magnesium, calcium, and barium, and particularly preferably calcium and barium.

The d-block element contained in the above zeolite preferably include scandium, titanium, vanadium, manganese, iron, cobalt, nickel, copper, zinc, yttrium, zirconium, niobium, molybdenum, ruthenium, rhodium, palladium, silver, cadmium, hafnium, tantalum, tungsten, rhenium, osmium, iridium, platinum and gold; more preferably vanadium, manganese, iron, cobalt, copper, niobium, molybdenum, silver, tantalum and tungsten; still more preferably manganese, iron, cobalt, copper and silver; further still more preferably manganese, copper and silver; and particularly preferably manganese and copper.

As the element contained in the above zeolite, alkali earth metal or a d-block element is preferred, and alkali earth metal is particularly preferred.

In the production of the cracking catalyst, as a method of introducing alkali metal, alkali earth metal and a d-block element in zeolite (preferably ZSM-5), a generally used method such as a method of introducing those after zeolite is produced, or a method of introducing those when zeolite is produced is properly selected. The method of introducing those when zeolite produced is preferred from the viewpoint of uniformly containing the metal, and the method of introducing those after zeolite is produced is preferred from the viewpoint that the commercially available zeolite can be easily used. Based on the type of zeolite to be used and easiness in maintenance of the production equipment using the production method of the present embodiment, a proper method can be selected to prepare a cracking catalyst.

Furthermore, the molar ratio of the element constituting the cracking catalyst can be acquired by Inductively Coupled Plasma Emission Spectrometry (hereinafter, also referred to as “ICP Spectrometry”)

(Production Method of Cracking Catalyst)

Hereinafter, the production method of the cracking catalyst used in the second process will be described.

In the case where alkali metal, alkali earth metal or transition metal (preferably a d-block element) are introduced when zeolite is produced, the cracking catalyst can be produced by preparing a mixture of a silicon source, an aluminum source, a structure regulating agent, a solvent and a raw material of the metal element (introduction element source), which is to be introduced to zeolite, in a pressure resistance vessel and by reacting them each other, thereby producing the cracking catalyst. The reaction temperature is preferably in the range of from 50° C. to 250° C., and more preferably in the range of from 100° C. to 200° C. Further, the reaction time is preferably in the range of from 0.1 hours to 150 hours, and more preferably in the range of from 1 hour to 120 hours.

Furthermore, a cracking catalyst can be produced by preparing dried gel which is dewatered from the above mixture in the pressure resistance vessel such that the dried gel is not allowed to come into contact with water or water containing the structure regulating agent, and by supplying vapor thereto to react them each other, thereby producing the cracking catalyst. The reaction temperature thereof is preferably in the range of from 50° C. to 250° C., and more preferably in the range of from 100° C. to 200° C. In addition, the reaction time thereof is preferably in the range of from 0.1 hours to 150 hours, and more preferably in the range of from 1 hour to 120 hours. Subsequently, a calcination treatment can be subjected to the obtained resultant with a predetermined temperature and time (for example, in the temperature range of from 300° C. to 800° C. for 1 hour to 48 hours)

The proper amount of the silicon source, the aluminum source, the structure regulating agent, and the introduction element source are reacted to each other such that the composition thereof is to have a target composition. In addition, only one kind may be used or two or more kinds may be used in combination thereof.

(Silicon Source)

The term “silicon source” represents a silicon-containing compound and a raw material that may become a component of zeolite of a cracking catalyst. The silicon source is not particularly limited as long as the raw material thereof may become a component of zeolite.

Examples of the silicon source include tetraalkyl orthosilicate, silica, silica gel, thermally decomposed silica, precipitated silica, colloidal silica, water glass, wet silica, amorphous silica, fumed silica, sodium silicate, kaolinite, diatomaceous earth and aluminum silicate, and preferably tetraalkyl orthosilicate and fumed silica.

(Aluminum Source)

The term “aluminum source” represents an aluminum-containing compound, and a raw material that may become a component of zeolite of a cracking catalyst. The aluminum source is not particularly limited as long as the raw material thereof may become a component of zeolite.

Examples of the aluminum source include aluminate, aluminum oxide, boehmite, aluminum oxyhydroxide, aluminum hydroxide, aluminum salts, (aluminum chloride, aluminum nitrate, and aluminum sulfate), aluminum alkoxide (aluminum isopropoxide and the like), alumina white, and aluminum fluoride, and preferably aluminum nitrate and aluminum oxide.

(Structure Regulating Agent)

The term a “structure regulating material” represents a compound for controlling a structure of zeolite. The structure regulating agent is not particularly limited, and various known structure regulating agents may be used. For example, an organic base, particularly a quaternary ammonium compound, and amine can be used therefor.

Specific examples of the structure regulating agent include, as a quaternary ammonium compound, a hydroxide salt such as tetramethylammonium, tetraethylammonium, tetrapropylammonium, tetra n-butylammonium, benzyltrimethylammonium, 3-(trifluoromethyl)phenyltrimethylammonium, and n-hexadecyltrimethylammonium, a phosphate, a fluoride salt, a chloride salt, a bromide salt and an acetate; and as amine, dipropylamine, triethylamine, cyclohexylamine, 1-methylamidazole, morpholine, pyridine, piperidine and diethylethanolamine.

Preferred examples of the structure regulating agent include a quaternary ammonium compound such as tetramethylammonium hydroxide, tetraethylammonium hydroxide, tetrapropylammonium hydroxide, tetra n-butylammonium hydroxide and benzyltrimethylammonium hydroxide; and amine such as dipropylamine, triethylamine, morpholine, pyridine and piperadine.

Further, more specific examples of the structure regulating agent include a quaternary ammonium compound such as tetramethylammonium hydroxide, tetraethylammonium hydroxide, tetrapropylammonium hydroxide, tetra n-butylammonium hydroxide and benzyltrimethylammonium hydroxide; and still more preferably tetrapropylammonium hydroxide.

(Introduction Element Source)

The term an “introduction element source” represents a compound containing one or more kinds of elements selected from the group consisting of alkali metal, alkali earth metal and transition metal which are introduced to zeolite of a cracking catalyst. Further, the introduction element source is not particularly limited as long as the element thereof may become a component of zeolite of a cracking catalyst, and examples thereof include a metal salt and a metal complex.

Specific examples of the introduction element source include a carbonate, a nitrate, a nitrite, a sulfate, a sulfite, an acetate, a formate, a phosphate, a hydrogenphosphate, a dihydrogen phosphate, a fluoride salt, a chloride salt, a bromide salt, an iodide salt, a hydroxide salt and an acetylacetonato complex, which are metal elements to be introduced. Among these, since anions can be easily removed by heating, a nitrate, a carbonate and an acetate are preferably used.

Moreover, metal elements in these compounds used for the introduction element source are one or more kinds of elements selected from the group consisting of alkali metal, alkali earth metal and a d-block element desirably.

Examples of the alkali metal contained in the introduction element source preferably include lithium, sodium, potassium, rubidium and cesium.

Examples of the alkali earth metal contained in the introduction element source preferably include beryllium, magnesium, calcium, strontium and barium, and more preferably magnesium, calcium, strontium and barium, still more preferably magnesium, calcium and barium, and particularly preferably calcium and barium.

The d-block element contained in the introduction element source preferably include scandium, titanium, vanadium, manganese, iron, cobalt, nickel, copper, zinc, yttrium, zirconium, niobium, molybdenum, ruthenium, rhodium, palladium, silver, cadmium, hafnium, tantalum, tungsten, rhenium, osmium, iridium, platinum and gold; more preferably vanadium, manganese, iron, cobalt, copper, niobium, molybdenum, silver, tantalum and tungsten; still more preferably manganese, iron, cobalt, copper and silver; further still more preferably manganese, copper and silver; and particularly preferably copper and manganese.

As the metal element contained in the introduction element source, alkali earth metal or a d-block element is preferred, and alkali earth metal is particularly preferred.

Further, specific examples of the introduction element source preferably include copper acetate, copper nitrate, manganese acetate, manganese nitrate, barium acetate, barium nitrate, calcium acetate and calcium nitrate, and more preferably barium acetate, barium nitrate, calcium acetate and calcium nitrate.

As the solvent used for introducing alkali metal, alkali earth metal and transition metal to the zeolite during the production of zeolite, a solvent which is generally used in the production of zeolite can be employed, and examples thereof include water, an alcohol compound, a nitrile compound, an amide compound, an aliphatic hydrocarbon, an alicyclic hydrocarbon, an aromatic hydrocarbon, an ether compound, a halogenated hydrocarbon and an ester compound. Among these, water, methanol, ethanol, propanol, ethylene glycol, acetonitrile, dimethylformamide, dimethylacetamide and N-methylpyrrolidone can be preferably used, and water, methanol, ethanol, propanol and ethylene glycol can be more preferably used. These solvents can be used as a mixture of the plural kinds thereof.

Further, a cracking catalyst can be produced by synthesizing zeolite using a mixture in which the introduction element source is excluded from the mixture obtainable in the method of producing the cracking catalyst described above, and introducing one or more kinds of metal elements selected from the group consisting of alkali metal, alkali earth metal and transition metal to the obtained zeolite. Subsequently, a calcination treatment can be subjected to the obtained resultant with a predetermined temperature and time (for example, at a temperature of from 300° C. to 800° C. for 1 hour to 48 hours).

As the method of introducing after zeolite is produced, a generally known method can be selected using a solution of salts containing the introduction metal elements. Specifically, the zeolite is immersed in the solution of salts containing introduction metal element, and then left to stand or stirred. At this time, the solution is left to stand or stirred in the temperature range of from 0° C. or more to less than 100° C., and preferably in the range of from 20° C. to 80° C. for from 0.1 hours to 24 hours, and preferably from 1 hour to 6 hours. In addition, the introduction metal element can be introduced to zeolite by carrying out either evaporation and drying or filtration and drying, on the obtained slurry. The evaporation and drying can be carried out in the temperature range of from 20° C. or more to less than 100° C., preferably in the range of from 40° C. to 80° C. for from 0.1 hour to 48 hours, and preferably from 1 hour to 24 hours. Further, in the filtration and drying, the solvent may be washed after the filtration, and then dried in the temperature range of from 20° C. to 150° C., preferably 60° C. to 130° C. for 1 hour to 48 hours and preferably 12 hours to 36 hours.

The introduction of the introduction metal element can be carried out multiple times if necessary, and the number of times of carrying out introduction is not particularly limited.

Specific examples of salts containing the introduction metal element include a carbonate, a nitrate, a nitrite, a sulfate, a sulfite, an acetate, a formate, a phosphate, a hydrogenphosphate, a dihydrogen phosphate, a fluoride salt, a chloride salt, a bromide salt, an iodide salt, a hydroxide salt and an acetylacetonato complex, which are salts or complexes of the introduction metal elements. Among these, since anions can be easily removed by heating, a nitrate and an acetate are preferably used.

The introduction metal element is desirably metal of which one or more kinds can be selected from the group consisting of alkali metal, alkali earth metal and a d-block element.

Examples of the alkali metal to be introduced preferably include lithium, sodium, potassium, rubidium and cesium.

Examples of the alkali earth metal to be introduced preferably include beryllium, magnesium, calcium, strontium and barium; and more preferably magnesium, calcium, strontium, and barium; still more preferably magnesium, calcium and barium; and particularly preferably calcium and barium.

The d-block element to be introduced preferably include scandium, titanium, vanadium, manganese, iron, cobalt, nickel, copper, zinc, yttrium, zirconium, niobium, molybdenum, ruthenium, rhodium, palladium, silver, cadmium, hafnium, tantalum, tungsten, rhenium, osmium, iridium, platinum and gold; more preferably vanadium, manganese, iron, cobalt, copper, niobium, molybdenum, silver, tantalum and tungsten; still more preferably manganese, iron, cobalt, copper, and silver; further still more preferably manganese, copper and silver; and particularly preferably copper and manganese.

In regard to the introduction metal element, alkali earth metal is more preferred.

The solution of salts containing the introduction element can be obtained by dissolving the above-described introduction element salt in a solvent.

Further, zeolite is immersed in the solution of salts containing the introduction metal element, and then left to stand or stirred. The introduction metal element can be introduced to the zeolite by carrying out either evaporation and drying or filtration and drying, on the obtained slurry.

Examples of the solvent, from the viewpoint of the high solubility of the introduction element salt, preferably include a polar solvent such as water, methanol, ethanol, propanol, ethylene glycol, acetonitrile, dimethylformamide, dimethylacetoamide, and N-methylpyrrolidone; more preferably water, methanol, ethanol, propanol, and ethylene glycol; and particularly preferably water. These solvents can be used as a mixture of the plural kinds thereof.

Since the impact of the performance degradation due to carbon deposition occurred by the side reaction of the catalytic cracking reaction is easily exerted as the particle size of the cracking catalyst is larger, therefore, the particle size thereof is preferably 5 μm or less, more preferably 3 μm or less, still more preferably in the range of from 0.01 μm to 2.5 μm, and particularly preferably in the range of from 0.01 μm to 2 μm.

The cracking catalyst may be used after performing a treatment such as pulverization, molding, or particle size regulation in advance.

The temperature range of the catalytic cracking process is preferably from 300° C. to 800° C., more preferably from 350° C. to 650° C., and still more preferably from 400° C. to 600° C.

The reaction pressure of the catalytic cracking is preferably in the range of from 0.01 MPa to 1 MPa, more preferably in the range of from 0.01 MPa to 0.5 MPa, and still more preferably in the range of from 0.05 MPa to 0.2 MPa.

In the case of continuous reaction, the time of the catalytic cracking process is represented by the ratio (V/F′) of the reaction volume (V) (mL) relative to the supply speed (F′) (mL/h) per volume of the hydrocarbon product obtained from the first process, and the ratio thereof is preferably in the range of from 1.0×10⁻⁶ h to 6 h, more preferably in the range of from 1.0×10⁻⁵ h to 3 h, and still more preferably in the range of from 1.0×10⁻⁴ h to 1 h.

A hydrocarbon product with the high content of olefin having 2 to 4 carbon atoms can be obtained from the above-described production method. In regard to the content of olefin having 2 to 4 carbon atoms, the ratio of the total carbon atoms constituting olefin having 2 to 4 carbon atoms relative to the total carbon atoms constituting the hydrocarbon product obtained from the above-described production method is preferably in the range of from 18% to 100%, more preferably in the range of from 24% to 100%, still more preferably in the range of from 30% to 100%, particularly preferably in the range of from 35% to 100%, and further still more preferably in the range of from 40% to 100%.

According to the production method described above, the content of olefin having 2 to 4 carbon atoms, particularly the content of propylene in the product can be enhanced.

From the viewpoint of the selectivity of olefin having 2 to 4 carbon atoms, particularly the selectivity of propylene in the product, the second embodiment is more preferable than the first embodiment.

### EXAMPLES

The present invention will be further described in detail by referring to the examples below, but the present invention is not limited thereto.

In the examples, obtained catalysts for the FT reaction and the results of the FT reaction are evaluated using the below-described analysis method.

(EDS Spectrometry)

The EDS spectrometry is performed using Energy Dispersive X-ray Fluorescence spectrometry equipment (RaynyEDX-700, manufactured by Shimadzu Corporation).

(Gas Chromatography)

In regard to the gas chromatography, Flame Ionization Detector (FID) measurement is performed using GC-14B and GC-2014AFsc (both manufactured by Shimadzu Corporation), and TCD (Thermal Conductivity Detector) measurement is performed using GC320 (manufactured by GL Sciences Inc.) and GC-2014AT (manufactured by Shimadzu Corporation).

(ICP Spectrometry)

The ICP spectrometry is performed using Inductively Coupled Plasma Emission spectrometry equipment (ICPE-9000, manufactured by Shimadzu Corporation).

Firstly, the first embodiment will be described.

### Example 1

Fe(NO₃)₃.9H₂O(20.2 g), Mn(NO₃)₂.6H₂O (2.2 g), and Cu(NO₃)₂.3H₂O (1.8 g) were weighed, and dissolved in water (300 ml) to prepare an Fe—Mn—Cu solution. Further, a 28% NH₄OH aqueous solution (80 ml) was weighed, and then water (420 ml) was added thereto to prepare an NH₄OH solution.

Water (300 ml) in a beaker was weighed and heated at 60° C., and then the above-described Fe—Mn—Cu solution was added dropwise to the water in a beaker over one hour while the water was stirred. At this time, the NH₄OH solution was added to the water in a beaker in advance to adjust the pH to about 8, and then the Fe—Mn—Cu solution was added dropwise to the water. Further, during the dropwise addition of the Fe—Mn—Cu solution, the NH₄OH solution was added dropwise while the pH of the reaction mixture was measured such that the pH of the reaction mixture in a beaker was maintained to about pH8.

After the completion of dropwise addition, the mixture was stirred for 1 hour, and the obtained reaction mixture was left to stand at the room temperature for 12 hours to cause precipitation.

The generated precipitation was filtrated and washed, and then dried at 120° C. for one night, thereby obtaining a dry matter. The obtained dry matter was pulverized using an agate mortar, and then a pulverized matter was obtained.

KNO₃ (0.126 g) was weighed, dissolved in water (3 g), and a solution of the KNO₃ was prepared to perform impregnation in accordance with the Incipient Wetness method. That is, the above-described pulverized matter was immersed in the obtained KNO₃ solution, and then the immersed matter was subjected to ultrasonication for 30 minutes.

Next, the obtained immersed matter was maintained at the room temperature for 1 hour under vacuum, and dried for one night at 120° C. under the normal pressure. Subsequently, the obtained dry matter was pulverized with the agate mortar.

The pulverized matter was introduced to an electric furnace in the atmosphere, and the temperature therein was increased from the room temperature to 400° C. for 80 minutes, and then maintained at 400° C. for 3 hours and subjected to the heat treatment thereto, thereby obtaining a catalyst 1.

From the result of the EDS analysis, the metal content (molar ratio) of the obtained catalyst 1 was Fe:Mn:Cu:K=74.3:11.2:12.5:2.0.

The catalyst 1 (1 g), and polyalphaolefin (20 ml, number average molecular weight of 735) were added to a reaction vessel with an inner capacity of 85 ml equipped with a stirrer. A synthesis gas of which the H₂/CO ratio was 0.97 was allowed to flow such that the ratio of the mass (W) (g) of the catalyst relative to the supply speed (F) (mol/h) of the synthesis gas (hereinafter, also referred to as the “W/F ratio”), was 10 g·h/mol, under the pressure of 0.1 MPa, and then an activation treatment was carried out at 300° C. for 10 hours. Here, the synthesis gas used for the FT reaction described later was used as reducing gas for the activation treatment.

Subsequently, the synthesis gas of which the H₂/CO ratio was 0.97 was allowed to flow with the W/F ratio of 10 g·h/mol under the pressure of 1 MPa, and then the FT reaction was carried out at 280° C. for 8 hours.

Furthermore, a CO conversion ratio, the selectivity of propylene, and the selectivity of C2 to C4 olefin were calculated by analyzing the product generated from the reaction using the gas chromatography.

### Example 2

Fe(NO₃)₃.9H₂O (20.2 g), Mn(NO₃)₂.6H₂O (2.2 g), and Cu(NO₃)₂.3H₂O (1.8 g) were weighed, and dissolved in water (300 ml) to prepare an Fe—Mn—Cu solution. Further, Na₂CO₃ (15 g) was weighed, and then water (300 ml) was added thereto to prepare an Na₂CO₃ solution.

Water (300 ml) and polyethylene glycol (31 ml, number average molecular weight of 300) in a beaker were weighed and heated at 60° C., and then the above-described Fe—Mn—Cu solution was added dropwise to the water and polyethylene glycol in a beaker over one hour while the water was stirred. At this time, the Na₂CO₃ solution was added to the water in a beaker in advance to adjust the pH to about 8, and then the Fe—Mn—Cu solution was added dropwise to the water. Further, during the dropwise addition of the Fe—Mn—Cu solution, the Na₂CO₃ solution was added dropwise while the pH of the reaction mixture was measured such that the pH of the reaction mixture in a beaker was maintained to about pH8.

After the completion of dropwise addition, the mixture was stirred for 1 hour, and the obtained reaction mixture was left to stand at the room temperature for 12 hours to cause precipitation.

The generated precipitation was filtrated and washed, and then dried at 120° C. for one night, thereby obtaining a dry matter. The obtained dry matter was pulverized with an agate mortar, and then a pulverized matter was obtained.

A catalyst 2 was obtained by carrying out the same procedures as Example 1 except that the pulverized matter and the KNO₃ solution prepared by weighing KNO₃ (0.126 g) and dissolving the KNO₃ in water (2.5 g) were used.

From the result of the EDS analysis, the metal content (molar ratio) of the obtained catalyst 2 was Fe:Mn:Cu:K=74.2:11.1:12.6:2.1.

The FT reaction was carried out in the same procedures as Example 1 except that the catalyst 2 (1 g) was used instead of the catalyst 1.

### Example 3

Fe(NO₃)₃.9H₂O (20.2 g) and Mn(NO₃)₂.6H₂O (4.39 g) were weighed, and dissolved in water (300 ml) to prepare an Fe—Mn solution. Further, Na₂CO₃ (16.5 g) was weighed, and then water (300 ml) was added thereto to prepare an Na₂CO₃ solution.

Water (300 ml) in a beaker was weighed and heated at 60° C., and then the above-described Fe—Mn solution was added dropwise to the water in a beaker over one hour while the water was stirred. At this time, the Na₂CO₃ solution was added to the water in a beaker in advance to adjust the pH to about 8, and then the Fe—Mn solution was added dropwise to the water. Further, during the dropwise addition of the Fe—Mn solution, the Na₂CO₃ solution was added dropwise while the pH of the reaction mixture was measured such that the pH of the reaction mixture in a beaker was maintained to about pH8.

After the completion of the dropwise addition, the mixture was stirred for 1 hour, and the obtained reaction mixture was left to stand at the room temperature for 12 hours to cause precipitation.

The generated precipitation was filtrated and washed, and then dried at 120° C. for one night, thereby obtaining a dry matter. The obtained dry matter was pulverized with an agate mortar, and then a pulverized matter was obtained.

A catalyst 3 was obtained by carrying out the same procedures as Example 1 except that the pulverized matter and the KNO₃ solution prepared by weighing KNO₃ (0.043 g) and dissolving the KNO₃ in water (3 g) were used.

From the result of the EDS analysis, the metal content (molar ratio) of the obtained catalyst 3 was Fe:Mn:K=74.5: 23.8:1.7 according to the results of the EDS analysis.

The FT reaction was carried out in the same procedures as Example 1 except that the catalyst 3 (1 g) was used instead of the catalyst 1.

### Example 4

Fe(NO₃)₃.9H₂O (20.2 g), Mn(NO₃)₂.6H₂O (2.2 g), and KNO₃ (0.126 g) were weighed, dissolved in ethylene glycol (20 ml), and then a 40% by mass ethanol aqueous solution (5 ml) was added thereto. Polymethacrylate (mixture of MX-500 (6 g) and MX-150 (12 g), both manufactured by Soken Chemical Engineering Co., Ltd.) was added thereto to be immersed for 5 hours thereby causing precipitation. The obtained precipitation was filtrated and dried at 120° C. for one night.

The dry matter was introduced to an electric furnace in the atmosphere, and the temperature therein was increased from the room temperature to 400° C. at a rate of temperature rise of 1° C./min, and then maintained at 400° C. for 6 hours and subjected to the heat treatment thereto, thereby obtaining a catalyst 4.

From the result of the EDS analysis, the metal content (molar ratio) of the obtained catalyst 4 was Fe:Mn:K=84.4:13.0:2.6.

The FT reaction was carried out in the same procedures as Example 1 except that the catalyst 4 (1 g) was used instead of the catalyst 1.

### Example 5

Fe(NO₃)₃.9H₂O (20.2 g) and Mn(NO₃)₂.6H₂O (4.39 g) were weighed, and dissolved in water (300 ml) to prepare an Fe—Mn solution. Further, (NH₄)₂CO₃ (11.5 g) was weighed, and then water (300 ml) was added thereto to prepare an (NH₄)₂CO₃ solution.

Water (300 ml) in a beaker was weighed and heated at 60° C., and then the above-described Fe—Mn solution was added dropwise to the water in a beaker over one hour while the water was stirred. At this time, the (NH₄)₂CO₃ solution was added to the water in a beaker in advance to adjust the pH to about 8, and then the Fe—Mn solution was added dropwise to the water. Further, during the dropwise addition of the Fe—Mn solution, the (NH₄)₂CO₃ solution was added dropwise while the pH of the reaction mixture was measured such that the pH of the reaction mixture in a beaker was maintained to about pH8.

After the completion of the dropwise addition, the mixture was stirred for 1 hour, and the obtained reaction mixture was left to stand at the room temperature for 12 hours to cause precipitation.

The generated precipitation was filtrated and washed, and then dried at 120° C. for one night, thereby obtaining a dry matter. Subsequently, the obtained dry matter was pulverized with an agate mortar, and then a pulverized matter was obtained.

A catalyst 5 was obtained by carrying out the same procedures as Example 1 except that the pulverized matter and the KNO₃ solution prepared by weighing KNO₃ (0.058 g) and dissolving the KNO₃ in water (2.5 g) were used.

The FT reaction was carried out in the same procedures as Example 1 except that the catalyst 5 (1 g) was used instead of the catalyst 1.

### Example 6

Fe(NO₃)₃.9H₂O (20.2 g), and Mn(NO₃)₂.6H₂O (4.39 g) were weighed, and dissolved in water (200 ml) to prepare an Fe—Mn solution. Further, a 28% NH₄OH aqueous solution (30.0 ml) was weighed, and then water (170 ml) was added thereto to prepare an NH₄OH solution.

Next, activated carbon (4 g, manufactured by Wako Pure Chemical Industries, Ltd.) and water (300 ml) in a beaker were weighed and heated at 60° C., and then the above-described Fe—Mn solution was added dropwise to the water in a beaker over one hour while the water was stirred. At this time, the NH₄OH solution was added to the water in a beaker in advance to adjust the pH to about 8, and then the Fe—Mn solution was added dropwise to the water. Further, during the dropwise addition of the Fe—Mn solution, the NH₄OH solution was added dropwise while the pH of the reaction mixture was measured such that the pH of the reaction mixture in a beaker was maintained to about pH8.

After the completion of the dropwise addition, the mixture was stirred for 1 hour, and the obtained reaction mixture was left to stand at the room temperature for 12 hours to cause precipitation.

The generated precipitation was filtrated and washed, and then dried at 120° C. for one night, thereby obtaining a dry matter. The obtained dry matter was pulverized with an agate mortar, and then a pulverized matter was obtained.

KNO₃ (0.086 g) was weighed, dissolved in water (10 g), and a solution of the KNO₃ was prepared to perform impregnation in accordance with the Incipient Wetness method. That is, the above-described pulverized matter was immersed in the obtained KNO₃ solution, and then the immersed matter was subjected to ultrasonication for 30 minutes.

Next, the obtained immersed matter was maintained at the room temperature for 1 hour under vacuum, and dried for one night at 120° C. The obtained dry matter was pulverized with the agate mortar.

Further, the pulverized matter was introduced to a tube furnace in the argon gas flow, and the temperature therein was increased from the room temperature to 400° C. for 80 minutes, and then maintained at 400° C. for 3 hours and subjected to the heat treatment thereto, thereby obtaining a catalyst 6.

From the result of the EDS analysis, the metal molar content of the obtained catalyst 6 was Fe:Mn:K=74.4:19.4:6.2.

The catalyst 6 (1 g), and polyalphaolefin (20 ml, Mn735) were added to a reaction vessel with an inner capacity of 85 ml equipped with a stirrer. Next, reducing gas of which the H₂/CO ratio was 0.67 was allowed to flow with the ratio of the mass (W) (g) of the catalyst relative to the supply speed (F) (mol/h) of the reducing gas, which was 10 g·h/mol, under the pressure of 0.1 MPa, and then an activation treatment was carried out at 280° C. for 10 hours.

Subsequently, the synthesis gas of which the H₂/CO ratio was 0.97 was allowed to flow with the W/F ratio of 10 g·h/mol under the pressure of 1 MPa, and then the FT reaction was carried out at 280° C. for 8 hours.

A CO conversion ratio, the selectivity of propylene, and the selectivity of C2 to C4 olefin were calculated by analyzing the product generated from the reaction using the gas chromatography.

### Example 7

Fe(NO₃)₃.9H₂O (40.43 g) and Mn(NO₃)₂.6H₂O (8.78 g) were weighed, and dissolved in water (300 ml) to prepare an Fe—Mn solution. Further, a 28% NH₄OH aqueous solution (90 ml) was weighed to prepare an NH₄OH solution.

Water (300 ml) in a beaker was weighed and heated at 60° C., and then the above-described Fe—Mn solution and the NH₄OH solution were added dropwise to the water in a beaker over one hour while the water was stirred. At this time, the NH₄OH solution was added to the water in a beaker in advance to adjust the pH to about 8, and then the Fe—Mn solution was added dropwise to the water. Further, during the dropwise addition of the Fe—Mn solution, the NH₄OH solution was added dropwise while the pH of the reaction mixture was measured such that the pH of the reaction mixture in a beaker was maintained to about pH8.

After the completion of the dropwise addition, the mixture was stirred for 30 hours, and the obtained reaction mixture was left to stand at the room temperature for 20 hours to cause precipitation.

The generated precipitation was filtrated and washed, and then dried at 120° C. for one night, thereby obtaining a dry matter. The obtained dry matter was pulverized with an agate mortar, and then a pulverized matter was obtained.

K₂CO₃ (0.0173 g) was weighed, dissolved in water (4.29 g), and a solution of the K₂CO₃ was prepared. That is, the above-described pulverized matter was dispersed in the obtained K₂CO₃ solution, and the dispersed solution was subjected to ultrasonication for 30 minutes and then stirred.

Next, the obtained mixture was maintained at the room temperature for 1 hour under vacuum, and dried for one night at 120° C. The obtained dry matter was pulverized with the agate mortar.

The pulverized matter was introduced to an electric furnace in the atmosphere, and the temperature therein was increased from the room temperature to 400° C. for 80 minutes, and then maintained at 400° C. for 3 hours and subjected to the heat treatment, thereby obtaining a catalyst 7 (11.32 g).

From the result of the EDS analysis, the metal molar content (molar ratio) of the obtained catalyst was Fe:Mn:K=75.47:22.64:1.89 according to the results of the EDS analysis.

The FT reaction was carried out in the same procedures as Example 1 except that the catalyst 7 (1 g) was used instead of the catalyst 1 and the FT reaction was carried out for 6 hours.

### Example 8

Fe(NO₃)₃.9H₂O (20.2 g) and Mn(NO₃)₂.6H₂O (6.46 g) were weighed, and dissolved in water (300 ml) to prepare an Fe—Mn solution. A catalyst 8 (6.2333 g) was obtained in the same procedures as Example 7 except that the prepared Fe—Mn solution was used.

From the result of the EDS analysis, the metal molar content (molar ratio) of the obtained catalyst was Fe:Mn:K=65.57:33.00:1.43.

The FT reaction was carried out in the same procedures as Example 1 except that the catalyst 8 (1 g) was used instead of the catalyst 1.

### Example 9

Fe(NO₃)₃.9H₂O (20.2 g) and Mn(NO₃)₂.6H₂O (8.79 g) were weighed, and dissolved in water (300 ml) to prepare an Fe—Mn solution. A catalyst 9 (6.87 g) was obtained in the same procedures as Example 7 except that the prepared Fe—Mn solution was used.

From the result of the EDS analysis, the metal molar content of the obtained catalyst was Fe:Mn:K=57.93:40.64:1.43.

The FT reaction was carried out in the same procedures as Example 7 except that the catalyst 9 (1 g) was used instead of the catalyst 1.

### Example 10

Fe(NO₃)₃.9H₂O (20.2 g) and Mn(NO₃)₂.6H₂O (10.76 g) were weighed, and dissolved in water (300 ml) to prepare an Fe—Mn solution. A catalyst 10 (6.87 g) was obtained in the same procedures as Example 7 except that the prepared Fe—Mn solution was used.

From the result of the EDS analysis, the metal molar content of the obtained catalyst was Fe:Mn:K=52.89:45.83:1.28.

The FT reaction was carried out in the same procedures as Example 7 except that the catalyst 10 (1 g) was used instead of the catalyst 1. The results are shown in Table 1 below.

### Example 11

Fe(NO₃)₃.9H₂O (20.2 g) and Mn(NO₃)₂.6H₂O (8.79 g) were weighed, and dissolved in water (300 ml) to prepare an Fe—Mn solution. Further, a 28% NH₄OH aqueous solution (45.0 ml) was weighed, and then water (300 ml) was added thereto to prepare an NH₄OH solution.

Powdered activated carbon (TAI KO S, manufactured by Futamura Chemical Co., Ltd 4 g) and water (200 ml) in a beaker were weighed and heated at 60° C., and then the above-described Fe—Mn solution and the NH₄OH solution were added dropwise to the water in a beaker over one hour while the water was stirred. At this time, the NH₄OH solution was added to the water in a beaker in advance to adjust the pH to about 8, and then the Fe—Mn solution was added dropwise to the water. Further, during the dropwise addition of the Fe—Mn solution, the NH₄OH solution was added dropwise while the pH of the reaction mixture was measured such that the pH of the reaction mixture in a beaker was maintained to about pH8.

After the completion of the dropwise addition, the mixture was stirred for 30 minutes, and the obtained reaction mixture was left to stand for 16 hours to cause precipitation.

The generated precipitation was filtrated and washed, and then dried at 120° C. for one night, thereby obtaining a dry matter. The obtained dry matter was pulverized with an agate mortar, and then a pulverized matter was obtained.

K₂CO₃ (0.086 g) was weighed, dissolved in water (20 g), and a solution of the K₂CO₃ was prepared. The above-described pulverized matter was dispersed in the obtained K₂CO₃ solution, and then the dispersed solution was subjected to ultrasonication for 30 minutes.

Next, the obtained mixture was maintained at the room temperature for 1 hour under vacuum, and dried for one night at 120° C. The obtained dry matter was pulverized with the agate mortar.

The pulverized matter was introduced to an electric furnace under the Ar atmosphere, and the temperature therein was increased from the room temperature to 400° C. for 80 minutes, and then maintained at 400° C. for 3 hours and subjected to the heat treatment thereto, thereby obtaining a catalyst 11 (10.17 g).

From the result of the EDS analysis, the metal molar content of the obtained catalyst was Fe:Mn:K=57.81:40.66:1.53.

The FT reaction was carried out in the same procedures as Example 7 except that the catalyst 11 (1 g) was used instead of the catalyst 1.

### Example 12

Fe(NO₃)₃.9H₂O (20.2 g) and Mn(NO₃)₂.6H₂O (8.79 g) were weighed, and dissolved to water (300 ml) to prepare an Fe—Mn solution. Further, a 28% NH₄OH aqueous solution (45.0 ml) was weighed, and then water (300 ml) was added thereto to prepare an NH₄OH solution.

Carbon black (Ketjenblack EC 600JD, manufactured by Lion Corporation, 4 g) and water (200 ml) in a beaker were weighed and heated at 60° C., and then the above-described Fe—Mn solution and the NH₄OH solution were added dropwise to the water in a beaker over one hour while the water was stirred. At this time, the NH₄OH solution was added to the water in a beaker in advance to adjust the pH to about 8, and then the Fe—Mn solution was added dropwise to the water. Further, during the dropwise addition of the Fe—Mn solution, the NH₄OH solution was added dropwise while the pH of the reaction mixture was measured such that the pH of the reaction mixture in a beaker was maintained to about pH8.

After the completion of dropwise addition, the mixture was stirred for 30 hours, and the obtained reaction mixture was left to stand for 16 hours to cause precipitation.

The generated precipitation was filtrated and washed, and then dried at 120° C. for one night, thereby obtaining a dry matter. The obtained dry matter was pulverized with an agate mortar, and then a pulverized matter was obtained.

K₂CO₃ (0.086 g) was weighed, dissolved in water (10 g), and a solution of the K₂CO₃ was prepared. The above-described pulverized matter was dispersed in the obtained K₂CO₃ solution, and then the dispersed solution was subjected to ultrasonication for 30 minutes.

Next, the obtained mixture was maintained at the room temperature for 1 hour under vacuum, and dried for one night at 120° C. The obtained dry matter was pulverized with the agate mortar.

The pulverized matter was introduced to an electric furnace in the Ar atmosphere, and the temperature therein was increased from the room temperature to 400° C. for 80 minutes, and then maintained at 400° C. for 3 hours and subjected to the heat treatment thereto, thereby obtaining a catalyst 12 (10.15 g).

From the result of the EDS analysis, the metal molar content of the obtained catalyst 12 was Fe:Mn:K=58.11:40.39:1.54.

The FT reaction was carried out in the same procedures as Example 7 except that the catalyst 12 (1 g) was used instead of the catalyst 1.

### Example 13

Fe(NO₃)₃.9H₂O (40.46 g) and Mn(NO₃)₂.6H₂O (17.23 g) were weighed, and dissolved in water (240 ml) to prepare an Fe—Mn solution. Further, a 28% NH₄OH aqueous solution (70 ml) was weighed to prepare an NH₄OH solution.

Water (240 ml) in a beaker was weighed and heated at 60° C., and then the above-described Fe—Mn solution and the above-described NH₄OH solution were added dropwise to the water in a beaker over 1.5 hours while the water was stirred. At this time, the NH₄OH solution was added to the water in a beaker in advance to adjust the pH to about 8, and then the Fe—Mn solution was added dropwise to the water. Further, during the dropwise addition of the Fe—Mn solution, the NH₄OH solution was added dropwise while the pH of the reaction mixture was measured such that the pH of the reaction mixture in a beaker was maintained to about pH8.

The mixture was stirred for 30 hours, and the obtained reaction mixture was left to stand at the room temperature for 16 hours to cause precipitation.

The generated precipitation was filtrated and washed, and then dried at 120° C. for one night, thereby obtaining a dry matter. The obtained dry matter was pulverized with an agate mortar, and then a pulverized matter was obtained.

Mg(NO₃)₂ (0.639 g) was weighed, dissolved in water (3 g), and a solution of the Mg(NO₃)₂ was prepared. That is, the above-described pulverized matter was dispersed in the obtained Mg(NO₃)₂ solution, and then the dispersed solution was subjected to ultrasonication for 30 minutes.

Next, the obtained mixture was maintained at the room temperature for 1 hour under vacuum, and dried for one night at 120° C. The obtained dry matter was pulverized with the agate mortar.

The pulverized matter was introduced to an electric furnace in the atmosphere, and the temperature therein was increased from the room temperature to 400° C. for 80 minutes, and then maintained at 400° C. for 3 hours and subjected to the heat treatment thereto, thereby obtaining a catalyst 13 (6.870 g) containing Fe, Mn, and Mg.

From the result of the EDS analysis, the metal molar content of Fe, Mn, and Mg in the obtained catalyst was Fe:Mn=59.57:40.11.

The FT reaction was carried out in the same procedures as Example 7 except that the catalyst 13 (1 g) was used instead of the catalyst 1.

### Example 14

The catalyst 9 (1 g), and polyalphaolefin (20 ml, number average molecular weight of 735) were added to a reaction vessel with an inner capacity of 85 ml equipped with a stirrer. A synthesis gas of which the H₂/CO ratio was 0.97 was allowed to flow with the W/F ratio of 10 g·h/mol, under the pressure of 0.1 MPa, and then an activation treatment was carried out at 300° C. for 10 hours. Here, the synthesis gas used for the FT reaction described below was used as reducing gas for the activation treatment.

Subsequently, the synthesis gas of which the H₂/CO ratio was 0.97 was allowed to flow with the W/F ratio of 10 g·h/mol under the pressure of 1 MPa, and then the FT reaction was carried out at 260° C. for 8 hours.

A CO conversion ratio, the selectivity of propylene, and the selectivity of C2 to C4 olefin were calculated by analyzing the product generated from the reaction using the gas chromatography.

The results of Examples 1 to 14 are shown in Table 1 below.

Moreover, in Table 1, the “conversion ratio” (%) is a ratio of the amount of CO (mole number) consumed in the FT reaction relative to the amount of CO (mole number) of a raw material to be used, the value determined by “[(consumed CO amount)/(raw material CO amount)]×100” (%) was adopted. In addition, the FT reaction was carried out using the catalyst after conducting the activation treatment, so that the conversion ratio was calculated relative to the ratio of CO amount in the mixture (i.e., the mixture of residues of raw materials and products) obtainable after the reaction.

Further, the “selectivity” (%) is either the ratio of amount of number of moles in carbon atoms contained in propylene or the ratio of amount of number of moles in carbon atoms contained in C2 to C4 olefin, relative to the number of moles of carbon atoms contained in the total hydrocarbon generated from the FT reaction.

As the selectivity of propylene, a value calculated by “[(amount of number of moles in carbon atoms contained in the generated propylene)/(amount of number of moles in carbon atoms contained in the generated total hydrocarbon)]×100” (%) was adopted.

Furthermore, as the selectivity of C2 to C4 olefin, a value calculated by “[(amount of number of moles in carbon atoms contained in the generated C2 to C4 olefin)/(amount of number of moles in carbon atoms contained in the generated total hydrocarbon)]×100” (%) was adopted.

TABLE 1

| | |
|---|---|
| Conversion ratio | Selectivity (%) |

| | (%) | Propylene | C2 to C4 olefin |
|---|---|---|---|
| Example 1 | 97 | 15 | 32 |
| Example 2 | 93 | 13 | 25 |
| Example 3 | 94 | 15 | 41 |
| Example 4 | 86 | 18 | 41 |
| Example 5 | 94 | 17 | 41 |
| Example 6 | 52 | 22 | 50 |
| Example 7 | 89 | 15 | 37 |
| Example 8 | 91 | 15 | 36 |
| Example 9 | 84 | 16 | 38 |
| Example 10 | 88 | 15 | 35 |
| Example 11 | 43 | 13 | 33 |
| Example 12 | 42 | 12 | 31 |
| Example 13 | 39 | 17 | 39 |
| Example 14 | 50 | 13 | 32 |

In this way, it was confirmed that the selectivities of C2 to C4 light olefins and propylenes were excellent in Examples 1 to 14. Particularly, it was confirmed that the selectivity of C2 to C4 light olefin and propylene in Example 6 was higher than those in Examples 1 to 5, and 7 to 14.

### Comparative Example 1

A fixed bed reactor was filled with the catalyst 7 (0.5 g), and synthesis gas (molar ratio of H₂:CO=1:1) was flowed at a flow rate of 40 mL/min at 300° C. under the normal pressure for 10 hours to carry out the activation treatment on the catalyst of the FT reaction. Subsequently, synthesis gas (molar ratio of H₂:CO=1:1) was flowed in the catalyst at a flow rate, in which W/F was 10 g·h/mol, at 300° C. under the pressure of 1 MPa to carry out the FT reaction. The reaction time was set to 6 hours. The obtained product was analyzed using the gas chromatography in the same manner as that of Example 1.

The results of Comparative Example 1 are shown in Table 2 below. For the comparison, the results of Example 7 are shown together.

Further, the “conversion ratio” (%) in Table 2 is the same as that of Table 1. The “selectivity” (%) in Table 2 is the ratio of amount of number of moles in carbon atoms contained in propylene or the ratio of amount of number of moles in carbon atoms contained in C2 to C4 olefin, relative to the number of moles in carbon atoms contained in the total hydrocarbon generated after the catalytic cracking.

TABLE 2

| | |
|---|---|
| Conversion ratio | Selectivity (%) |

| | (%) | Propylene | C2 to C4 olefin |
|---|---|---|---|
| Example 7 | 89 | 15 | 37 |
| Comparative | 38 | 9 | 27 |
| Example 1 | | | |

In this way, it was confirmed that Example 7 using a dispersion medium was high in the selectivity of C2 to C4 light olefin and propylene as compared with Comparative Example 1 in which a dispersion medium was not used.

The usefulness of the first embodiment was confirmed from the results above.

Next, the second embodiment will be described.

### Example 15

Tetraethyl orthosilicate (TEOS) (2.227 g) was gradually added to a solution containing a 10% tetrapropylammonium hydroxide aqueous solution (6.506), Al(NO₃)₃.9H₂O (0.029 g), Ba (CH₃COO)₂ (0.097 g), ion exchange water (8.555 g) and ethanol (1.968 g), and the solution was intensively stirred to form a uniform sol, and subjected to hydrothermal synthesis at 180° C. for 24 hours to cause precipitation.

The obtained precipitation was filtrated and washed, and then dried at 120° C. to obtain a dry matter. The obtained dry matter was calcinated at 550° C. in the atmosphere for 5 hours, thereby obtaining 0.617 g of a cracking catalyst 1.

From the result of the ICP analysis, the oxide content (molar ratio) of the obtained cracking catalyst 1 was SiO₂/Al₂O₃/BaO=252/1.00/6.97.

The below-described reaction was carried out using a production apparatus including a slurry bed reactor with an inner capacity of 85 mL equipped with a stirrer, a fixed bed reactor connected to the slurry bed reactor interposing a pipe therebetween, and a back pressure valve provided on the pipe interposed between the slurry bed reactor and the fixed bed reactor. The slurry bed reactor is a reactor performing the first process (FT reaction) of the present invention, the fixed bed reactor is a reactor performing the second process (catalytic cracking reaction) of the present invention, and they correspond to the production apparatus shown in FIG. 1.

The above-described catalyst 9 (1.0 g) and polyalphaolefin (20 ml, number average molecular weight of 735) were added to the slurry bed reactor. Synthesis gas of which the H₂/CO ratio was 0.97 was allowed to flow with the W/F ratio of 10 g·h/mol, under the pressure of 0.1 MPa, and then an activation treatment was carried out at 300° C. for 10 hours.

Subsequently, the synthesis gas of which the H₂/CO ratio was 0.97 was allowed to flow with the W/F ratio of 20 g·h/mol under the pressure of 1.0 MPa, and then the FT reaction was carried out at 280° C. for 6 hours to synthesize a hydrocarbon compound.

The generated hydrocarbon compound was allowed to pass through the back pressure valve maintained at 100° C. and to flow to the fixed bed reactor filled with the catalyst (0.3 g) which was prepared in the same manner as that of the above-described cracking catalyst 1. In the fixed bed reactor, catalytic cracking was carried out at 550° C. under the normal pressure to obtain a cracked product. The catalytic cracking was started simultaneously at the time of the FT reaction, and the treating time thereof was set to 6 hours.

A gas component and a liquid component were separately collected from the cracked product by passing through the ice-cooled trap, and these components were analyzed using the gas chromatography.

### Example 16

TEOS (2.225 g) was gradually added to a solution containing a 10% tetrapropylammonium hydroxide aqueous solution (6.504 g), Al(NO₃)₃.9H₂O (0.028 g), Ba (CH₃COO)₂ (0.051 g), ion exchange water (8.574 g) and ethanol (1.978 g), and the solution was intensively stirred to form a uniform sol, and subjected to hydrothermal synthesis at 180° C. for 24 hours to cause precipitation. The obtained precipitate was calcinated in the same manner as that of Example 15, thereby obtaining 0.555 g of a cracking catalyst 2.

From the result of the ICP analysis, the oxide content (molar ratio) of the obtained cracking catalyst 2 was SiO₂/Al₂O₃/BaO=278/1.00/5.37.

A cracked product was obtained in the same manner as that of Example 15 except that the flow rate (W/F ratio) of the synthesis gas at the time of synthesizing the hydrocarbon compound after the activation treatment in the first process was set to 10 g·h/mol and the reaction temperature in the catalytic cracking reaction was set to 500° C. using the catalyst prepared in the same manner as the cracking catalyst 2 in the second process. The cracked product was analyzed using the gas chromatography which was the same manner as that of Example 15.

### Example 17

TEOS (2.240 g) was gradually added to a solution containing a 10% tetrapropylammonium hydroxide aqueous solution (6.506 g), Al(NO₃)₃.9H₂O (0.028 g), Ba (CH₃COO)₂ (0.020 g), ion exchange water (8.574 g) and ethanol (1.968 g), and the solution was intensively stirred to form a uniform sol, and then subjected to hydrothermal synthesis at 180° C. for 24 hours to cause precipitation. The obtained precipitate was calcinated in the same manner as that of Example 15, thereby obtaining 0.598 g of a cracking catalyst 3. From the result of the ICP analysis, the oxide content (molar ratio) of the obtained cracking catalyst 3 was SiO₂/Al₂O₃/BaO=253/1.00/1.74.

A cracked product was obtained in the same manner as that of Example 15 except that the flow rate (W/F ratio) of the synthesis gas during the synthesis of the hydrocarbon compound after the activation treatment in the first process was set to 10 g·h/mol and the catalyst prepared in the same manner as that of the cracking catalyst 3 in the second process was used. The cracked product was analyzed using the gas chromatography which was the same manner as that of Example 15.

### Example 18

Tetraethyl orthosilicate (2.250 g) was gradually added to a solution containing a 10% by mass tetrapropylammonium hydroxide aqueous solution (6.507 g), aluminum nitrate nonahydrate (0.029 g), barium acetate (0.010 g), ion exchange water (8.544 g) and ethanol (1.968 g) and the solution was intensively stirred to form a uniform sol, and then hydrothermal synthesis was carried out at 180° C. for 24 hours. The obtained precipitate was dried at 120° C. and calcinated at 550° C. for 5 hours, thereby obtaining 0.603 g of a cracking catalyst 4.

From the result of the ICP analysis, the oxide content (molar ratio) of the obtained cracking catalyst 4 was SiO₂/Al₂O₃/BaO=306/1.00/1.05.

A cracked product was obtained in the same manner as that of Example 17 except that the catalyst obtained in the same manner as the above-described cracking catalyst 4 was used instead of cracking catalyst 3. The cracked product was analyzed using the gas chromatography which was the same manner as that of Example 17.

### Example 19

2.232 g of tetraethyl orthosilicate was gradually added to a solution containing a 10% by mass tetrapropylammonium hydroxide aqueous solution (6.507 g), aluminum nitrate nonahydrate (0.029 g), manganese nitrate hexahydrate (0.022 g), ion exchange water (8.546 g) and ethanol (1.973 g), and the solution was intensively stirred to form a uniform sol, and then subjected to hydrothermal synthesis at 180° C. for 24 hours. The obtained precipitate was dried at 120° C. and calcinated at 550° C. for 5 hours, thereby obtaining 0.550 g of a cracking catalyst 5.

From the result of the ICP analysis, the oxide content (molar ratio) of the obtained cracking catalyst 5 was SiO₂/Al₂O₃/MnO₂=287/1.00/0.86.

A cracked product was obtained in the same manner as that of Example 17 except that the catalyst obtained in the same manner as the above-described cracking catalyst 5 was used instead of the cracking catalyst 3. The cracked product was analyzed using the gas chromatography which was the same manner as that of Example 17.

### Example 20

After 0.492 g of HZSM-5 (SiO₂/Al₂O₃=280) was impregnated to a solution in which 0.022 g of copper nitrate trihydrate was dissolved with 5.005 g of deionized water, and the resultant was dried and calcinated at 600° C. for 5 hours, thereby obtaining 0.438 g of a cracking catalyst 6.

From the result of the ICP analysis, The oxide content (molar ratio) of the obtained cracking catalyst 6 was SiO₂/Al₂O₃/CuO=267/1.00/1.11.

A cracked product was obtained in the same manner as that of Example 17 except that the catalyst obtained in the same manner as the above-described cracking catalyst 6 was used instead of the cracking catalyst 2. The cracked product was analyzed using the gas chromatography which was the same manner as that of Example 17.

### Example 21

After 2.006 g of HZSM-5 (SiO₂/Al₂O₃=280) was impregnated with a solution in which 0.053 g of calcium acetate monohydrate was dissolved in 20.189 g of deionized water, and the resultant was dried and calcinated at 600° C. for 5 hours, thereby obtaining 1.845 g of a cracking catalyst 7.

From the result of the ICP analysis, the oxide content (molar ratio) of the obtained cracking catalyst 7 was SiO₂/Al₂O₃/CaO=297/1.00/2.28.

A cracked product was obtained in the same manner as that of Example 17 except that the catalyst obtained in the same manner as the above-described cracking catalyst 7 was used instead of the cracking catalyst 2. The cracked product was analyzed using the gas chromatography which was the same manner as that of Example 17.

### Example 22

After 2.019 g of HZSM-5 (SiO₂/Al₂O₃=280) was impregnated with a solution in which 0.078 g of magnesium nitrate hexahydrate was dissolved in 20.052 g of deionized water, and the resultant was dried and calcinated at 600° C. for 5 hours, thereby obtaining 1.979 g of a cracking catalyst 8.

From the result of the ICP analysis, the oxide content (molar ratio) of the obtained cracking catalyst 8 was SiO₂/Al₂O₃/CaO=299/1.00/2.76.

A cracked product was obtained in the same manner as that of Example 17 except that the catalyst obtained in the same manner as the above-described cracking catalyst 8 was used instead of cracking catalyst 2. The cracked product was analyzed using the gas chromatography which was the same manner as that of Example 17.

### Example 23

After 0.500 g of the catalyst obtained in the same manner as the cracking catalyst 4 was impregnated with a solution in which 0.071 g of ammonium hydrogenphosphate was dissolved in 4.998 g of deionized water, and the resultant was dried and calcinated at 600° C. for 4 hours, thereby obtaining 0.368 g of a cracking catalyst 9.

From the result of the ICP analysis, the oxide content (molar ratio) of the obtained cracking catalyst 9 was SiO₂/Al₂O₃/BaO/P₂O₅=246/1.00/0.57/5.36.

A cracked product was obtained in the same manner as that of Example 17 except that the catalyst obtained in the same manner as the above-described cracking catalyst 9 was used instead of cracking catalyst 2. The cracked product was analyzed using the gas chromatography which was the same manner as that of Example 17.

### Example 24

Fe(NO₃)₃.9H₂O (40.41 g), Co(NO₃)₂.6H₂O (29.11 g), and Mn(NO₃)₂.6H₂O (28.71 g) were weighed, and dissolved in water (140 ml) to prepare an Fe—Co—Mn solution. Further, Na₂CO₃ (42.40 g) was weighed, and dissolved in water (200 ml) to prepare an Na₂CO₃ solution.

Next, the above described Fe—Co—Mn solution was moved to a beaker and heated at 60° C., and then the Na₂CO₃ solution was added dropwise to the Fe—Co—Mn solution in a beaker over two hours while the solution was stirred. Further, during the dropwise addition of the Na₂CO₃ solution, the Na₂CO₃ solution was added dropwise while the pH of the reaction mixture was measured such that the pH of the reaction mixture in a beaker was maintained to about pH8 to pH10.

After the completion of the dropwise addition, the mixture was stirred for 0.5 hours, and the obtained reaction mixture was left to stand for 2 hours to cause precipitation.

The generated precipitation was filtrated and washed, and then dried at 120° C. for 12 hours, thereby obtaining a dry matter. The obtained dry matter was pulverized with an agate mortar, and then a pulverized matter was obtained.

The pulverized matter was introduced to an electric furnace in the atmosphere, and the temperature therein was increased from the room temperature to 600° C. for 2.5 hours, and then maintained at 600° C. for 6 hours and subjected to the heat treatment thereto, thereby obtaining a catalyst 14 (21.47 g).

From the result of the EDS analysis, the metal molar content of the obtained catalyst was Fe:Co:Mn=34.56:32.85:32.57.

A silica tube was filled with the above-described catalyst 14 (1.5 g), hydrogen gas (80 ml/min) was flowed therethrough as reducing gas, and the silica tube was treated at 400° C. for 10 hours, and then was cooled to the room temperature while nitrogen gas was flowed therethrough. Further, gas (15 ml/min) of which the ratio of oxygen:argon was 0.01 was flowed in the silica tube for 4 hours. In this way, a catalyst 14 which was subjected to hydrogenation was obtained.

The catalyst 14 (1.0 g), which was produced by the above-described method and subjected to hydrogenation, and polyalphaolefin (20 ml, number average molecular weight of 735) were added to the slurry bed reactor. Next, synthesis gas of which the H₂/CO ratio was 0.97 was allowed to flow therein under the condition of 0.1 MPa and 40 ml/min, and then the resultant was maintained at 240° C. for 1 hour. Furthermore, synthesis gas of which the H₂/CO ratio was 0.97 was allowed to flow with the W/F ratio of 10 g·h/mol under the pressure of 1 MPa, and then the resultant was treated at 240° C. for 6 hours.

Subsequently, the synthesis gas of which the H₂/CO ratio was 0.97 was allowed to flow with the W/F ratio of 10 g·h/mol under the pressure of 1 MPa, and then the FT reaction was carried out at 280° C. for 6 hours.

The generated hydrocarbon compound was allowed to pass through the back pressure valve maintained at 100° C. and to flow to the fixed bed reactor filled with the catalyst (0.3 g) which was prepared in the same manner as that of the above-described cracking catalyst 4. In the fixed bed reactor, catalytic cracking was carried out at 550° C. under the normal pressure to obtain a cracked product. The catalytic cracking was started simultaneously at the time of the FT reaction, and the treating time thereof was set to 6 hours.

A gas component and a liquid component were separately collected from the cracked product by passing through the ice-cooled trap, and the components thereof were analyzed using the gas chromatography.

### Example 25

The temperature of CARiACT Q-50 (manufactured by Fuji Silysia Chemical, Ltd. 5.5 g) was increased from the room temperature to 300° C. in the atmosphere over 1 hour, and was maintained at 300° C. for 2 hours. The CARiACT Q-50 was moved to an evaporating dish, a zirconia dispersion liquid (2.25 g, ZR-30BFN, manufactured by Nissan Chemical Industries, Ltd. 30.5% by mass of solid content of zirconia) was impregnated thereto in accordance with the Incipient Wetness method.

The temperature of the impregnated material was increased from the room temperature to 600° C. in the atmosphere for 1 hour, and was maintained at 600° C. for 2 hours. The impregnated material which was subjected to the heat treatment was moved to the evaporating dish, a Co solution (prepared by dissolving Co(NO₃)₂.6H₂O (3.54 g) in water (4.20 g)) was impregnated thereto with the Incipient Wetness method.

The impregnated material was dried at 120° C. in the atmosphere over 12 hours, and the temperature thereof was increased from the room temperature to 400° C. in the atmosphere over 3 hours to be maintained at 400° C. for 2 hours, and then the impregnated material was subjected to the heat treatment, thereby obtaining a catalyst 15 (6.93 g).

The metal molar content of the obtained catalyst was Co:Zr=26.59:9.43.

A silica tube was filled with the above-described catalyst 15 (1.5 g), hydrogen gas (40 ml/min) was flowed therethrough as reducing gas, and the silica tube was treated at 400° C. for 10 hours, and then was cooled to the room temperature while nitrogen gas was flowed therethrough. Further, gas (15 ml/min) of which the ratio of oxygen/argon was 0.01 was flowed in the silica tube for 4 hours. In this way, a catalyst 15 which was subjected to hydrogenation was obtained.

The catalyst 15 (1 g), which was prepared by the above-described method and subjected to the hydrogenation, and hexadecane (20 ml) were added to a reaction vessel with an inner capacity of 85 ml equipped with a stirrer. Next, reducing gas of which the H₂/CO ratio was 2 was allowed to flow therein under the condition of 40 ml/min and 0.1 MPa, and then the resultant was maintained at 240° C. for 1 hour. Subsequently, synthesis gas of which H₂/CO ratio was 2 was allowed to flow with the W/F ratio of 10 g·h/mol under the pressure of 0.5 MPa, and then the FT reaction was carried out at 220° C. for 6 hours.

The generated hydrocarbon compound was allowed to pass through the back pressure valve maintained at 100° C. and to flow to the fixed bed reactor filled with the catalyst (0.3 g) which was prepared in the same manner as that of the above-described cracking catalyst 4. In the fixed bed reactor, catalytic cracking was carried out at 550° C. under the normal pressure to obtain a cracked product. The catalytic cracking was started simultaneously at the time of the FT reaction, and the treating time thereof was set to 6 hours.

A gas component and a liquid component were separately collected from the cracked product by passing through the ice-cooled trap, and the components thereof were analyzed using the gas chromatography.

The results of Examples 15 to 25 of the second embodiment, and Example 9 of the first embodiment are shown in Table 3 below.

Further, the “conversion ratio” (%) in Table 3 is the same as that of Table 1. The “selectivity” (%) in Table 3 is either the ratio of amount of number of moles in carbon atoms contained in propylene or the ratio of amount of number of moles in carbon atoms contained in C2 to C4 olefin, relative to the number of moles in carbon atoms contained in the total hydrocarbon generated after the catalytic cracking.

TABLE 3

| | |
|---|---|
| Conversion ratio | Selectivity (%) |

| | (%) | Propylene | C2 to C4 olefin |
|---|---|---|---|
| Example 15 | 86 | 22 | 50 |
| Example 16 | 91 | 28 | 55 |
| Example 17 | 81 | 29 | 58 |
| Example 18 | 86 | 31 | 61 |
| Example 19 | 86 | 31 | 62 |
| Example 20 | 77 | 27 | 55 |
| Example 21 | 81 | 29 | 55 |
| Example 22 | 75 | 30 | 56 |
| Example 23 | 77 | 23 | 44 |
| Example 24 | 66 | 16 | 29 |
| Example 25 | 15 | 17 | 31 |
| Example 9 | 84 | 16 | 38 |

Accordingly, it is understood that the selectivities of C2 to C4 light olefins and propylenes of Examples 15 to 23 in which the catalytic cracking was carried out are much higher than those of Example 9 in which the FT reaction conditions were equivalent but the catalytic cracking was not carried out.

The usefulness of the second embodiment was further confirmed from the results above.

### INDUSTRIAL APPLICABILITY

According to the production method of the present invention, C2 to C4 light olefin, particularly propylene can be selectively produced by the FT reaction.

### REFERENCE SIGNS LIST

1 . . . tank, 2 . . . first reactor, 3 . . . back pressure valve, 4 . . . second reactor

1. A method of producing olefin having 2 to 4 carbon atoms, comprising a process of reacting at least one kind of a catalyst (D) selected from the group consisting of catalysts (A) to (C) with synthesis gas in the presence of a dispersion medium through a Fischer-Tropsch reaction,
wherein the catalyst (A) contains iron and one to three kinds of elements selected from the group consisting of alkali metal and alkali earth metal,
the catalyst (B) contains cobalt, provided that the catalyst (B) is a catalyst except a catalyst obtained by reducing a cobalt ion and an iron ion in a dispersion liquid or a solution containing the cobalt ion, the iron ion and a dispersant that interacts with the cobalt ion and the iron ion, and
the catalyst (C) contains nickel or ruthenium.

2. The method of producing olefin having 2 to 4 carbon atoms according to claim 1, wherein the catalyst (D) contains one to three kinds of elements selected from the group consisting of manganese, copper, zinc, titanium, zirconium, lanthanum and cerium.

3. The method of producing olefin having 2 to 4 carbon atoms according to claim 1,
wherein the catalyst (D) contains elements (1) and elements (2), and satisfies a condition (3),
the elements (1) are iron and manganese,
the elements (2) are one to three kinds of metal elements selected from the group consisting of alkali metal and alkali earth metal, and
the condition (3) is 50≦a≦90, 9.5≦b≦48, and 0.5≦c≦10, provided that a+b+c=100, when the molar ratio of iron is represented by a mole %, the molar ratio of manganese is represented by b mole %, and the molar ratio of the total metal elements in the elements (2) is represented by c mole %, relative to the total number of moles of the iron, the manganese and the elements (2).

4. The method of producing olefin having 2 to 4 carbon atoms according to claim 1, wherein the catalyst (D) further contains a carbon support.

5. The method of producing olefin having 2 to 4 carbon atoms according to claim 1,
wherein the synthesis gas contains hydrogen and carbon monoxide, and
the molar ratio of the hydrogen relative to the carbon monoxide, which is represented by “hydrogen/carbon monoxide”, is in the range of from 0.3 to 3.

6. The method of producing olefin having 2 to 4 carbon atoms according to claim 1, wherein the reaction temperature in the process of reacting the synthesis gas with the catalyst (D) is in the range of from 100° C. to 600° C.

7. The method of producing olefin having 2 to 4 carbon atoms according to claim 1, wherein the reaction pressure in the process of reacting the synthesis gas with the catalyst (D) is in the range of from 0.1 MPa to 50 MPa.

8. The method of producing olefin having 2 to 4 carbon atoms according to claim 1, wherein the dispersion medium is an organic compound which becomes a liquid state in the temperature range of from 100° C. to 600° C. under the normal pressure.

9. The method of producing olefin having 2 to 4 carbon atoms according to claim 1, wherein the ratio of the total number of carbon atoms constituting olefin having 2 to 4 carbon atoms relative to the total number of carbon atoms constituting a hydrocarbon product obtained from the process of reacting the synthesis gas with the catalyst (D) is 18% or more.

10. The method of producing olefin having 2 to 4 carbon atoms according to claim 1, further comprising a process of catalytically cracking the product obtained from the process of reacting the synthesis gas with the catalyst (D), after the process of reacting the synthesis gas with the catalyst (D).

11. A method of producing propylene which uses the method of producing olefin having 2 to 4 carbon atoms according to claim 1.

12. A method of producing olefin having 2 to 4 carbon atoms, comprising:
a first process of reacting synthesis gas and a catalyst (E) in the presence of a dispersion medium to produce a hydrocarbon product through a Fischer-Tropsch reaction; and
a second process of catalytically cracking the hydrocarbon product by allowing the hydrocarbon product to come into contact with a cracking catalyst which is consisting of zeolite containing one or more kinds of elements selected from the group consisting of alkali metal, alkali earth metal, and transition metal.

13. The method of producing olefin having 2 to 4 carbon atoms according to claim 12, wherein the zeolite contains one or more kinds of elements selected from the group consisting of alkali metal, alkali earth metal, and a d-block element.

14. The method of producing olefin having 2 to 4 carbon atoms according to claim 12,
wherein the zeolite is ZSM-5, and
the molar ratio of SiO₂ relative to Al₂O₃ in the zeolite, which is represented by “SiO₂/Al₂O₃”, is in the range of from 50 to 4000.

15. The method of producing olefin having 2 to 4 carbon atoms according to claim 12, wherein the cracking catalyst contains one or more kinds of elements selected from the group consisting of the alkali metal, the alkali earth metal and the transition metal of which the content of the elements is in the range of from 0.01% by mass to 30% by mass relative to the total mass of the cracking catalyst.

16. The method of producing olefin having 2 to 4 carbon atoms according to claim 12, wherein one or more kinds of elements selected from the group consisting of the alkali metal, the alkali earth metal, and the transition metal contained in the cracking catalyst is alkali earth metal.

17. The method of producing olefin having 2 to 4 carbon atoms according to claim 12, wherein the reaction pressure in the catalytic cracking is in the range of from 0.01 MPa to 0.5 MPa.

18. The method of producing olefin having 2 to 4 carbon atoms according to claim 12, wherein the catalyst (E) contains at least one kind of element selected from the group consisting of iron, cobalt, nickel, and ruthenium.

19. The method of producing olefin having 2 to 4 carbon atoms according to claim 18, wherein the catalyst (E) further contains one to three kinds of elements selected from the group consisting of manganese, copper, zinc, titanium, zirconium, lanthanum and cerium.

20. The method of producing olefin having 2 to 4 carbon atoms according to claim 18, wherein the catalyst (E) further contains one to three kinds of elements selected from the group consisting of alkali metal and alkali earth metal.

21. The method of producing olefin having 2 to 4 carbon atoms according to claim 12,
wherein the catalyst (E) contains elements (1) and elements (2), and satisfies a condition (3),
the elements (1) are iron and manganese,
the elements (2) are one to three kinds of metal elements selected from the group consisting of alkali metal and alkali earth metal, and
the condition (3) is 50≦a≦90, 9.5≦b≦48, and 0.5≦c≦10, provided that a+b+c=100, when the molar ratio of iron is represented by a mole %, the molar ratio of manganese is represented by b mole %, and the molar ratio of the total metal elements in the elements (2) is represented by c mole %, relative to the total number of moles of the iron, the manganese and the elements (2).

22. The method of producing olefin having 2 to 4 carbon atoms according to claim 12, wherein the catalyst (E) further contains a carbon support.

23. The method of producing olefin having 2 to 4 carbon atoms according to claim 12,
wherein the synthesis gas contains hydrogen and carbon monoxide, and
the molar ratio of the hydrogen relative to the carbon monoxide, which is represented by “hydrogen/carbon monoxide”, is in the range of from 0.3 to 3.

24. The method of producing olefin having 2 to 4 carbon atoms according to claim 12, wherein the reaction temperature in the first process is in the range of from 100° C. to 600° C.

25. The method of producing olefin having 2 to 4 carbon atoms according to claim 12, wherein the reaction pressure in the first process is in the range of from 0.1 MPa to 50 MPa.

26. The method of producing olefin having 2 to 4 carbon atoms according to claim 12, wherein the dispersion medium is an organic compound which becomes a liquid state in the temperature range of from 100° C. to 600° C. under the normal pressure.

27. The method of producing olefin having 2 to 4 carbon atoms according to claim 12, wherein the ratio of the total number of carbon atoms constituting olefin having 2 to 4 carbon atoms relative to the total number of carbon atoms constituting the hydrocarbon product obtained from the first process is 18% or more.

28. A method of producing propylene which uses the method of producing olefin having 2 to 4 carbon atoms according to claim 12.

29. A method of producing olefin having 2 to 4 carbon atoms, comprising a process of reacting synthesis gas with a catalyst which contains at least one kind of element selected from the group consisting of iron, cobalt, and nickel and contains one to three kinds of elements selected from the group consisting of alkali metal and alkali earth metal, in the presence of a dispersion medium through a Fischer-Tropsch reaction.
